(19) United States
(12) Patent Application Publication
(10) Pub. No.: US 2007/0015205 A1
(43) Pub. Date: Jan. 18, 2007

(54) Crystal structure of phosphodiesterase 5 and use thereof

(76) Inventors: David Graham Brown, Sandwich (GB); Colin Roger Groom, Kent (GB); Andrew Lee Hopkins, Kent (GB); Timothy Mark Jenkins, Kent (GB); Sarah Helen Kamp, Kent (GB); Margaret Mary O'Gara, Cedex (GB); Heather Joan Ringrose, Kent (GB); Colin Mark Robinson, Kent (GB); Wendy Elaine Taylor, Kent (GB)

Correspondence Address: PFIZER INC., PATENT DEPARTMENT, MS8260-1611, EASTERN POINT ROAD, GROTON, CT, 06340, US

(21) Appl. No.: 10/415,839

(22) PCT Filed: Oct. 24, 2002
(86) PCT No.: PCT/IB02/04426

§ 371 Date: Oct. 27, 2003

(30) Foreign Application Priority Data
Nov. 2, 2001 (GB) 012641705

(51) Int. Cl. G01N 33/53; C12N 9/16

(52) U.S. Cl. 435/7.1; 435/196

(57) ABSTRACT

The present invention relates, inter alia, to the crystal structures of a phosphodiesterase 5 (PDE5) and PDE5/PDE5 ligand complex and their uses in identifying PDE5 ligands, including PDE5 inhibitor compounds. The present invention also relates to methods of identifying such PDE5 inhibitor compounds and their medical use. Also contemplated by the present invention are crystals of PDE5/PDE5 inhibitor complexes.

### TECHNICAL FIELD

The present invention relates to the crystal structures of a phosphodiesterase 5 (PDE5) and PDE5/PDE5 ligand complex and their uses in identifying PDE5 ligands, including PDE5 inhibitor compounds. The present invention also relates to methods of identifying such PDE5 inhibitor compounds and their medical use. Also contemplated by the present invention are crystals of PDE5/PDE5 inhibitor complexes.

### BACKGROUND OF THE INVENTION

A wide variety of biological processes, including cardiac muscle contraction, regulation of blood flow, neural transmission, glandular secretion, cell differentiation and gene expression are affected by steady state levels of the cyclic nucleotide biological second messengers cAMP and cGMP. Intracellular receptors for these molecules include cyclic nucleotide dependent protein kinases (PGK) (Lohmann et al. 1997), cyclic nucleotide-gated channels, and class I phosphodiesterases (PDEs) (Charbonneau 1990). PDEs are a large family of proteins, which were first reported by Sutherland and co-workers (Rall & Sutherland 1958, Butcher & Sutherland 1962). The family of cyclic nucleotide phosphodiesterases catalyse the hydrolysis of 3′,5′-cyclic nucleotides to the corresponding 5′ monophosphates. Current literature shows that there are eleven related, but biochemically distinct, human phosphodiesterase gene groups and that many of these groups include more than one gene subtype giving a total of twenty genes. Some PDEs are highly specific for hydrolysis of cAMP (PDE4, PDE7, PDE8), some are highly cGMP specific (PDE5, PDE6, PDE9), and some have mixed specificity (PDE1, PDE2, PDE3, PDE10, PDE11).

All PDEs are multi-domain proteins; each PDE has a ˜270 amino acid domain located towards the C-terminus, which has a high degree of amino acid sequence conservation between families (Charbonneau 1986). This domain has been extensively studied and shown to be responsible for the common catalytic function (Francis, S. H. et al. 1994). Non-homologous segments in the remainder of the protein have regulatory function or confer specific binding properties. PDE2, PDE5, PDE6 and PDE10 are all reported to contain putative GAF domains within their regulatory amino terminal portion (Aravind & Ponting 1997 and Soderling & Beavo 2000). These GAF domains have been shown to bind cGMP but their function is not yet fully understood. Full length mammalian PDEs characterised to date are dimeric in solution, but the relevance of the dimeric structure is unknown. The structure of the regulatory segment of PDE2A bound to cGMP has recently been solved and reveals a parallel dimer of four GAF domains, with cGMP binding to only one of the two GAF domains on each monomer (Martinez, et al. 2001).

PDE5, a cGMP specific PDE, has been recognised in recent years as an important therapeutic target. It is composed of the conserved C-terminal, zinc containing, catalytic domain, which catalyses the cleavage of cGMP, and an N-terminal regulatory portion, which contains two GAF domain repeats. Each GAF domain contains a cGMP-binding site, one of high affinity and the other of lower affinity. PDE5 activity is regulated through binding of cGMP to the high and low affinity cGMP binding sites followed by phosphorylation, which occurs only when both sites are occupied (Thomas et al. 1990). PDE5 is found in varying concentrations in a number of tissues including platelets, vascular and visceral smooth muscle, and skeletal muscle. The protein is a key regulator of cGMP levels in the smooth muscle of the erectile corpus cavernosal tissue. The physiological mechanism of erection involves release of nitric oxide (NO) in the corpus cavernosum during sexual stimulation. NO then activates the enzyme guanylate cyclase, which results in increased levels of cGMP, producing smooth muscle relaxation in the corpus cavernosum and allowing in flow of blood. Inhibition of PDE5 inhibits the breakdown of cGMP allowing the levels of cGMP, and hence smooth muscle relaxation, to be maintained (Corbin & Francis 1999). Sildenafil (UK-092,480), the active ingredient of Viagra® and a potent inhibitor of PDE5, has attracted widespread attention for the effective treatment of male erectile dysfunction.

Structural information has recently been shown for the catalytic domain of PDE4b a cAMP-specific PDE (Xu et al. 2000). This structure provides information about the overall fold of the catalytic domains of the PDE family, but, to date, no structural information is known about the way in which potential inhibitors bind to the enzyme.

The X-ray structure of a recombinant construct of PDE5 comprising the catalytic domain in complex with Sildenafil has been determined. An engineered form of this construct, which shows improved qualities for the production of crystals of PDE5/inhibitor complexes, has also been produced. This protein has also been used to solve its structure bound to Sildenafil. These complexes not only provide important structural information on this novel family of proteins but will also assist the design of more potent and specific inhibitors to treat the many diseases where PDEs play a role.

### SUMMARY OF THE INVENTION

It has been found that PDE5 can be crystallised. It has also been found that manipulating the wild-type PDE5 amino acid sequence can facilitate the crystallisation of PDE5. Specifically, it has been found that manipulations of certain portions of the PDE5 amino acid sequence can facilitate the crystallisation of PDE5.

It has been shown that manipulations of the catalytic domain of PDE5, specifically the 657-682 region of PDE5 (the “loop region”), can facilitate the crystallisation of PDE5. More specifically, manipulations of the loop region amino acid sequence (HRGVNNSYIQRSEHPLAQLYCHSIME=SEQ ID NO: 1) of PDE5 can facilitate the crystallisation of PDE5. This manipulation can be achieved by deletion, addition or substitution of one or more amino acid residues of the PDE5 loop region or it can be achieved by complete replacement of the PDE5 loop region with a loop region (or other equivalent amino acid sequence, e.g. sub-domain) from another protein, preferably another PDE, more preferably PDE4, most preferably PDE4b.

Crystals of PDE5 have been found to be useful for screening for PDE5 ligands, especially PDE5 inhibitors (e.g. by co-crystallising PDE5 with the PDE5 ligand (e.g. PDE5 inhibitor) or by soaking the PDE5 ligand (e.g. PDE5 inhibitor) into the crystal of PDE5).

PDE5 ligands, especially PDE5 inhibitors, as identified by the methods of the present invention are useful in curative, palliative or prophylactic treatments.

Thus, the present invention provides the following (numbered) aspects:

1. A crystal of phosphodiesterase 5 (PDE5).

2. The crystal of PDE5 according to aspect 1, wherein said PDE5 is from a mammal.

3. The crystal of PDE5 according to aspect 1 or aspect 2, wherein said PDE5 is from a human.

4. The crystal of PDE5 according to any one of aspects 1 to 3, wherein said PDE5 is an isoform selected from the group consisting of PDE5A1, PDE5A2, PDE5A3 and PDE5A4.

5. The crystal of PDE5 according to aspect 3 or aspect 4, wherein said PDE5 comprises SEQ ID NO: 1 or a homologue, fragment, variant, analogue or derivative thereof.

SEQ ID NO: 1 is the so-called “loop region” of PDE5. This loop region or a homologue, fragment, variant, analogue or derivative thereof includes additions, deletions or substitutions of amino acid residues comprised within the loop region.

Preferably, a variant in relation to the amino acid sequence of the crystal of the PDE5 of the present invention includes the deletion or substitution of the histidine (His/H) residue as shown emboldened and underlined in SEQ ID NO: 1 (HRGVNNSYIQRSEHPLAQLYCHSIME). This histidine co-ordinates a zinc atom in wild-type PDE5. Replacement of said histidine (H) residue is preferably by way of incorporating one or more amino acid residues (other than histidine), preferably wherein said amino acid residues are neutral or non-polar.

More preferably, a variant in relation to the amino acid sequence of the crystal of the PDE5 of the present invention includes the complete replacement of the loop region with a loop region (or other amino acid sequence e.g. an equivalent sub-domain) from another protein, preferably a PDE, more preferably PDE4, most preferably PDE4b (see hereinafter).

Alternatively, a variant in relation to the amino acid sequence of the crystal of the PDE5 of the present invention includes the deletion or substitution of the amino acid residues PLAQ (proline, leucine, alanine and glutamine) as emboldened and underlined in SEQ ID NO: 1 (HRGVNNSYIQRSEHPLAQLYCHSIME). The amino acid sequence PLAQ represents a proteolytic cleavage site of PDE5. By manipulating this site, e.g. by deleting and/or substituting one or more of the amino acid residues, undesired proteolytic cleavage of PDE5 can be lessened or prevented. Preferably, such substitution of amino acid residues utilises amino acids of similar charge to those substituted.

Manipulations of the “loop region” of PDE5 can be carried out in accordance with the present invention to stabilise the region. Similar manipulations may be carried out in PDE5-related proteins, other PDEs and PDE-related proteins in order to stablise such proteins.

The present invention further provides the following (numbered) aspects:

6. The crystal of PDE5 according to any one of aspects 3 to 5, wherein said PDE5 comprises SEQ ID NO: 2 or a homologue, fragment, variant, analogue or derivative thereof. Preferably, said PDE5 consists of SEQ ID NO: 2 or a homologue, fragment, variant, analogue or derivative thereof.

7. The crystal of PDE5 according to any one of aspects 3 to 6, wherein said PDE5 comprises SEQ ID NO: 3 or a homologue, fragment, variant, analogue or derivative thereof. Preferably, said PDE5 consists of SEQ ID NO: 3 or a homologue, fragment, variant, analogue or derivative thereof.

8. The crystal of PDE5 according to aspect 3 or aspect 4, wherein said PDE5 comprises SEQ ID NO: 4 or a homologue, fragment, variant, analogue or derivative thereof.

SEQ ID NO: 4 is the so-called “loop region” (or sub-domain) of PDE4 (PDE4b). This loop region or a homologue, fragment, variant, analogue or derivative thereof includes additions, deletions or substitutions of amino acid residues comprised within the loop region.

9. The crystal of PDE5 according to any one of aspects 3, 4 or 8, wherein said PDE5 comprises SEQ ID NO: 5 or a homologue, fragment, variant, analogue or derivative thereof. Preferably, said PDE5 consists of SEQ ID NO: 5 or a homologue, fragment, variant, analogue or derivative thereof.

10. The crystal of PDE5 according to any one of aspects 3, 4, 8 or 9, wherein said PDE5 comprises SEQ ID NO: 6 or a homologue, fragment, variant, analogue or derivative thereof. Preferably, said PDE5 consists of SEQ ID NO: 6 or a homologue, fragment, variant, analogue or derivative thereof.

11. A crystal of a PDE5/PDE5 ligand complex.

12. The crystal of a PDE5/PDE5 ligand complex according to aspect 11, wherein said PDE5 ligand is a PDE5 inhibitor.

13. The crystal of a PDE5/PDE5 ligand complex according to aspect 12, wherein said PDE5 inhibitor is Sildenafil.

14. The crystal of a PDE5/PDE5 ligand complex according to any one of aspects 11 to 13, wherein said PDE5 is as defined in any one of aspects 1 to 10.

15. The crystal of a PDE5/PDE5 ligand complex according to any one of aspects 11 to 13, wherein said PDE5 is as defined in any one of aspects 5 to 7.

16. The crystal of a PDE5/PDE5 ligand complex according to any one of aspects 11 to 13, wherein said PDE5 is as defined in any one of aspects 8 to 10.

17. The crystal of PDE5 according to any one of aspects 1 to 10 or the crystal of the PDE5/PDE5 ligand complex according to any one of aspects 11 to 16, which is grown in a solution containing buffer and/or precipitant.

18. The crystal of PDE5 according to any one of aspects 5 to 7, which is grown in a solution containing buffer and/or phosphate.

19. The crystal of PDE5 according to aspect 18, wherein said phosphate buffer is sodium/potassium phosphate, sodium phosphate or ammonium phosphate. Preferably, said phosphate buffer is 1.8-2.3M sodium phosphate at pH 3.4-5.0, with or without 0.1M Hepes pH 7.0-8.0, or 1.8-2.3M sodium/potassium phosphate at pH 3.4-5.0, with or without 0.1M Hepes pH 7.0-8.0.

20. The crystal of PDE5 according to any one of aspects 8 to 10 or the crystal of the PDE5/PDE5 ligand complex according to any one of aspects 11 to 16, which is grown in a solution containing:
(i) Tris or MES buffer, ammonium phosphate and/or PEG2KMME. Preferably, said Tris or MES buffer is at pH 6.0-8.4. More preferably, said solution contains 0.1 M Tris, pH 8.0, 50 mM ammonium phosphate, pH 7.0; 16-26% w/v PEG2KMME. Alternatively, said solution contains 0.1 M MES pH 6.0-6.5, 50 mM ammonium phosphate, pH 7.5; 22-34% w/v PEG2KMME; or
(ii) 0.16M Sodium Acetate, 80 mM Tris hydrochloride pH 8.5, 24% w/v Polyethylene Glycol 8000 (or PEG8KMME).

21. The crystal of PDE5 according to any one of aspects 8 to 10 or the crystal of a PDE5/PDE5 ligand complex according to aspect 16, which is grown in a solution containing:
(i) Tris buffer, sodium acetate and/or PEG4K. Preferably, said Tris buffer is at pH 6.5-8.6. Alternatively, said Tris buffer is at pH 8.2-8.6. More preferably, said solution contains 0.1M Tris pH 8.2-8.6, 0.2M sodium acetate and 26-30% w/v PEG4K; or
(ii) 0.16M Sodium Acetate, 80 mM Tris hydrochloride pH 8.5, 24% w/v Polyethylene Glycol 8000 (or PEG8KMME).

22. The crystal of PDE5 as defined in any one of the aspects 5 to 7, which has one or more of the following characteristics:
(a) a space group P6₂;
(b) unit cell dimensions a˜95 Å±1%, b˜95 Å±1%, c˜82 Å±1%, α=β=90°, γ=120°;
(c) 1 molecule per asymmetric unit;
(d) comprises a PDE5 of a molecular weight of approximately 40 kDa±2 kDa;
(e) a calculated solvent content of approximately 43±5%; and
(f) a hexagonal crystal system.

23. The crystal of the PDE5/PDE5 ligand complex according to aspect 15, which has one or more of the following characteristics:
(a) a space group P2₁2₁2₁;
(b) unit cell dimensions a˜94 Å±1%, b˜104 Å±1%, c˜142 Å±1%, α=β=γ=90°;
(c) 4 molecules per asymmetric unit;
(d) comprises a PDE5 of a molecular weight of approximately 40 kDa±2 kDa;
(e) a calculated solvent content of approximately 43±5%; and
(f) an orthorhombic crystal system.

24. The crystal of PDE5 as defined in any one of aspects 8 to 10 or the crystal of the PDE5/PDE5 ligand complex according to aspect 16, which has one or more of the following characteristics:
(a) a space group P2₁;
(b) unit cell dimensions a ˜55Å±1%, b˜78 Å±1%, c˜82 Å±1%, α=γ=90, β˜101°±2°;
(c) 2 molecules per asymmetric unit;
(d) comprises a PDE5 of a molecular weight of approximately 38 kDa±2 kDa;
(e) a calculated solvent content of approximately 46±5%; and
(f) a monoclinic crystal system.

25. The crystal of PDE5 according to any one of aspects 5 to 7, wherein said PDE5 has a three-dimensional structure characterised by the atomic co-ordinates set out in Table 3 or a derivative set as expressed in any reference frame.

26. The crystal of the PDE5/PDE5 ligand complex according to aspect 15, wherein said PDE5/PDE5 ligand complex has a three-dimensional structure characterised by the atomic co-ordinates set out in Table 4 or a derivative set as expressed in any reference frame.

27. The crystal of PDE5 according to any one of aspects 8 to 10, wherein said PDE5 has a three-dimensional structure characterised by the atomic co-ordinates set out in Table 5 or a derivative set as expressed in any reference frame.

28. The crystal of the PDE5/PDE5 ligand complex according to aspect 16, wherein said PDE5/PDE5 ligand complex has a three-dimensional structure characterised by the atomic co-ordinates set out in Table 6 or a derivative set as expressed in any reference frame.

29. Use of the atomic co-ordinates determined from the crystal of PDE5 according to aspect 25 or the crystal of the PDE5/PDE5 ligand complex according to aspect 26 for deriving a three-dimensional structure of (i) a full-length wild-type PDE5 or a mutant, derivative, fragment, variant, analogue or homologue thereof or (ii) a wild-type PDE5 sub-domain or a mutant, derivative, fragment, variant, analogue or homologue thereof.

30. Use according to aspect 29, wherein said PDE5 sub-domain is the catalytic domain.

31. Use of the atomic co-ordinates determined from the crystal of PDE5 according to aspect 27 or the crystal of the PDE5/PDE5 ligand complex according to aspect 28 for deriving a three-dimensional structure of (i) a full-length wild-type PDE5 or a mutant, derivative, fragment, variant, analogue or homologue thereof or (ii) a wild-type PDE5 sub-domain or a mutant, derivative, fragment, variant, analogue or homologue thereof.

32. Use according to aspect 31, wherein the PDE5 sub-domain is the catalytic domain.

33. Use of the three-dimensional structure of (i) a full-length wild-type PDE5 or a mutant, derivative, fragment, variant, analogue or homologue thereof or (ii) a wild-type PDE5 sub-domain or a mutant, derivative, fragment, variant, analogue or homologue thereof, as derivable according to any one of aspects 29, 30, 31 or 32 to computationally or otherwise evaluate the binding interactions of a PDE5 ligand with an active site on PDE5.

34. Use according to aspect 33, wherein said PDE5 ligand is a PDE5 inhibitor.

35. Use according to aspect 34, wherein said PDE5 inhibitor is Sildenafil.

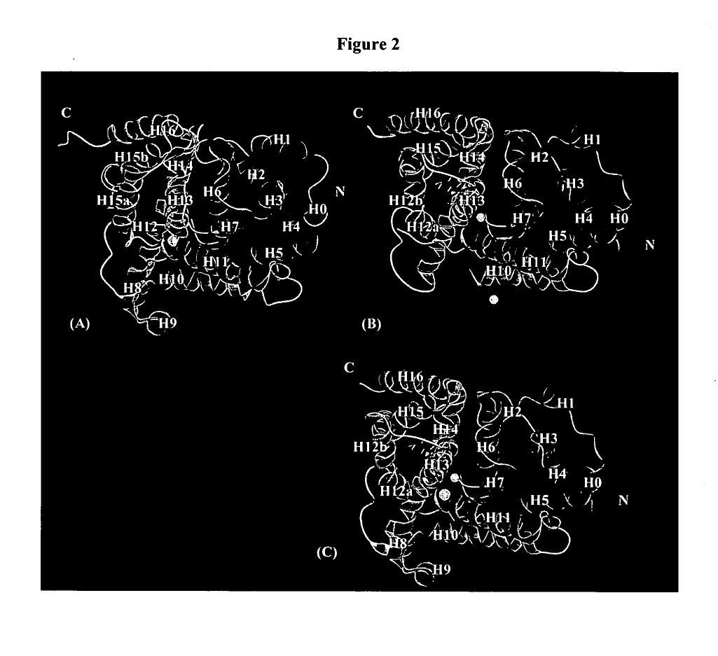

36. Use according to any one of aspects 33 to 35, wherein said active site on PDE5 is within the third sub-domain of the protein and is bounded by Helices 15 (H15 813-824) and 14 (H14 772-797), the C-terminus of Helix 13 (H13 749-765), and the C-terminus of Helix 11 (H11 706-721) along with the loop region between Helices 11 and 12a (H12a 725-731) as shown in FIG. 2.

37. Use according to any one of aspects 33 to 36, wherein said active site on PDE5 comprises Leu 765, Ala 767 and Ile 768 and one or more of Phe 820, Val 782, Phe 786, Tyr 612, Leu 804, Ala 779, Ala 783, Ile 813, Met 816 and Gln 817.

38. Use according to any one of aspects 33 to 37 to design a compound capable of associating with PDE5.

39. Use according to any one of aspects 33 to 38 to design a compound capable of associating with any active site of PDE5.

40. Use according to aspect 38 or aspect 39, wherein said compound is a PDE5 ligand.

41. Use according to aspect 40, wherein said PDE5 ligand is a PDE5 inhibitor.

42. A method of identifying a compound capable of associating with PDE5, comprising co-crystallising or soaking said compound with the crystal of PDE5 according to any one of aspects 1 to 10 and determining the three-dimensional structure to ascertain whether said compound is bound to PDE5. With respect to “soaking”, it should be noted that the compound can be added to the crystal, and thus the compound is soaked into the crystal. Alternatively, the crystal can be added to the compound (e.g. in solution), and again the compound is soaked into the crystal.

43. A method of identifying a compound capable of associating with any active site of PDE5, comprising co-crystallising or soaking said compound with the crystal of PDE5 according to any one of aspects 1 to 10 and determining the three-dimensional structure to ascertain whether said compound is bound to an active site of PDE5.

44. A compound designed by the use according to any one of aspects 33 to 41 or identified by the method of aspect 42 or aspect 43.

45. The compound according to aspect 44, which is a PDE5 inhibitor.

46. A method of selecting a PDE5 ligand from a group of potential PDE5 ligands, comprising the following steps:
(a) computationally creating a three-dimensional representation of the structure of PDE5 as derived from the atomic co-ordinates as defined in any one of aspects 29, 30, 31 or 32, and a three-dimensional representation of the structure of the potential PDE5 ligand;
(b) co-displaying the three-dimensional representation of the potential PDE5 ligand with the three-dimensional representation of the PDE5 structure; and
(c) assessing whether the three-dimensional representation of the potential PDE5 ligand fits the three-dimensional representation of an active site of the PDE5 structure.

47. The method according to aspect 46, further comprising the following steps:
(d) incorporating the potential PDE5 ligand in a biological PDE5 activity assay; and
(e) determining whether the potential PDE5 ligand modulates PDE5 activity in said assay.

48. The method according to aspect 46 or aspect 47 wherein said potential PDE5 ligand is a potential PDE5 inhibitor compound and said potential PDE5 inhibitor compound inhibits PDE5 activity.

49. A PDE5 ligand selected by the method of aspect 46 or aspect 47 or a PDE5 inhibitor compound selected by the method of aspect 48.

50. A pharmaceutical composition comprising one or more PDE5 ligands or PDE5 inhibitor compounds according to aspect 49 and one or more pharmaceutically acceptable excipients.

51. Use of a PDE5 ligand or PDE5 inhibitor compound according to aspect 49 as a pharmaceutical.

52. Use of a PDE5 ligand or PDE5 inhibitor compound according to aspect 49 in the manufacture of a medicament for the prophylaxis or treatment of a condition, disease, disorder or dysfunction where the inhibition of PDE5 is prophylactically or therapeutically beneficial.

53. Use according to aspect 52, wherein said disorder is a mammalian sexual disorder. The curative, palliative or prophylactic treatments contemplated by the present invention include the curative, palliative or prophylactic treatment of mammalian sexual disorders, in particular the treatment of mammalian sexual dysfunctions such as male erectile dysfunction (MED), impotence, female sexual dysfunction (FSD), clitoral dysfunction, female hypoactive sexual desire disorder, female sexual arousal disorder (FSAD), female sexual pain disorder or female sexual orgasmic dysfunction (FSOD) as well as sexual dysfunction due to spinal cord injury or selective serotonin re-uptake inhibitor (SSRI) induced sexual dysfunction but, clearly, will also be useful for treating other medical conditions for which PDE5 inhibitor is indicated. Such conditions include premature labour, dysmenorrhoea, benign prostatic hyperplasia (BPH), bladder outlet obstruction, incontinence, stable, unstable and variant (Prinzmetal) angina, hypertension, pulmonary hypertension, chronic obstructive pulmonary disease, coronary artery disease, congestive heart failure, atherosclerosis, conditions of reduced blood vessel patency, e.g. post-percutaneous transluminal coronary angioplasty (post-PTCA), peripheral vascular disease, stroke, nitrate induced tolerance, bronchitis, allergic asthma, chronic asthma, allergic rhinitis, diseases and conditions of the eye such as glaucoma, optic neuropathy, macular degeneration, elevated intra-occular pressure, retinal or arterial occlusion and diseases characterised by disorders of gut motility, e.g. irritable bowel syndrome (IBS).

Further medical conditions for which a PDE5 inhibitor is indicated, and for which treatment with compounds of the present invention may be useful include pre-eclampsia, Kawasaki's syndrome, multiple sclerosis, diabetic nephropathy, neuropathy including autonomic and peripheral neuropathy and in particular diabetic neuropathy and symptoms thereof e.g. gastroparesis, peripheral diabetic neuropathy, Alzheimer's disease, acute respiratory failure, psoriasis, skin necrosis, cancer, metastasis, baldness, nutcracker oesophagus, anal fissure, haemorrhoids, the insulin resistance syndrome, diabetes, hypoxic vasoconstriction as well as the stabilisation of blood pressure during haemodialysis.

Particularly preferred conditions include MED and FSD (preferably FSAD).

Further (numbered) aspects of the present invention include:

54. Use of the atomic co-ordinates determined from the crystal of PDE5 as defined in aspect 25 or aspect 27 or the crystal of the PDE5/PDE5 ligand complex as defined in aspect 26 or aspect 28, to solve the crystal structure of a mutant, derivative, fragment, variant, analogue, homologue or complex of a PDE-related protein.

55. Use according to aspect 54, wherein said PDE-related protein is a PDE.

56. Use according to aspect 55, wherein said PDE is a PDE5-related protein.

57. Use according to aspect 56, wherein said PDE5-related protein is PDE5.

58. Use of the atomic co-ordinates determined from the crystal of PDE5 as defined in aspect 25 or aspect 27 or the crystal of the PDE5/PDE5 ligand complex as defined in aspect 26 or aspect 28, to produce a model of the three-dimensional structure of a PDE-related protein.

59. Use according to aspect 58, wherein said PDE-related protein is a PDE.

60. Use according to aspect 59, wherein said PDE is a PDE5-related protein.

61. Use according to aspect 60, wherein said PDE5-related protein is PDE5.

62. Use of the three-dimensional structure of PDE5 as derivable as set out in any one of aspects 29, 30, 31 or 32 to design site-directed mutants that mimic other PDE5 isoforms or variants thereof.

63. A crystal of PDE5 or a crystal of a PDE5/PDE5 ligand complex wherein the active site on PDE5 is within the third sub-domain of the protein and is bounded by Helices 15 (H15 813-824) and 14 (H14 772-797), the C-terminus of Helix 13 (H13 749-765), and the C-terminus of Helix 11 (H11 706-721) along with the loop region between Helices 11 and 12a (H12a 725-731) as shown in FIG. 2.

64. A crystal of PDE5 or a crystal of a PDE5/PDE5 ligand complex wherein the active site on PDE5 comprises Leu 765, Ala 767 and Ile 768 and one or more of Phe 820, Val 782, Phe 786, Tyr 612, Leu 804, Ala 779, Ala 783, Ile 813, Met 816 and Gln 817.

65. A crystal of PDE5 wherein the crystal system of said crystal is characterised as being monoclinic, orthorhombic or hexagonal.

66. A crystal of a PDE5/PDE5 ligand complex wherein the crystal system of said crystal is characterised as being monoclinic or orthorhombic.

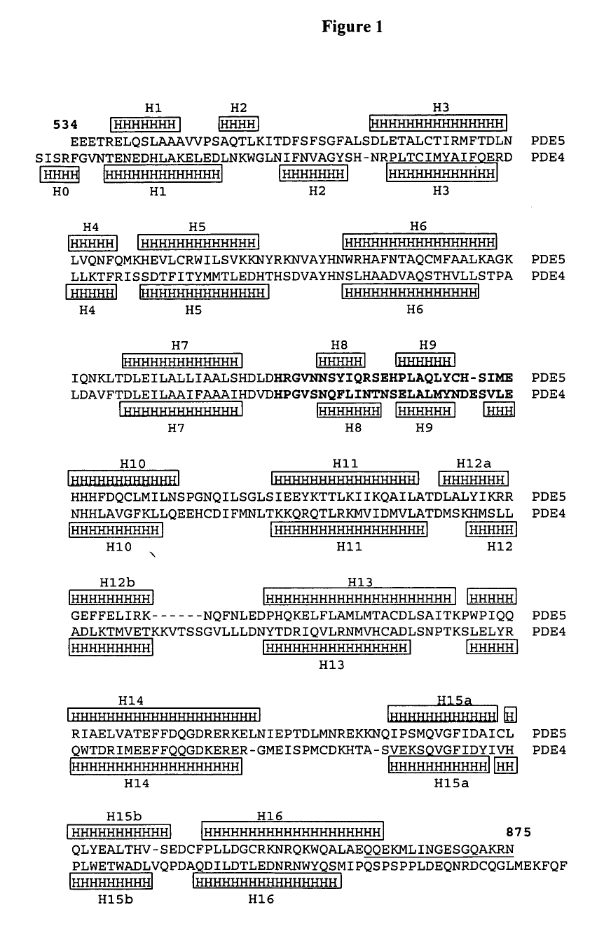

67. A method of producing a structurally stabilised PDE-related protein, comprising:
(a) aligning the amino acid sequence of a PDE-related protein with the amino acid sequence of (i) PDE4, (ii) the catalytic domain of PDE4 as shown in FIG. 1 or (iii) SEQ ID NO: 4;
(b) identifying the structurally equivalent sub-domain of the PDE-related protein that corresponds to SEQ ID NO: 4; and
(c) genetically engineering the replacement of the structurally equivalent sub-domain of the PDE-related protein or a portion thereof with SEQ ID NO: 4 or a homologue, fragment, variant, analogue or derivative thereof.

68. The method according to aspect 67, further comprising:
(d) expressing the engineered PDE-related protein from (c) in a host cell.

69. The method according to aspect 68, further comprising:
(e) purifying the expressed engineered PDE-related protein from (d).

70. The method according to aspect 69, further comprising:
(f) crystallising the purified engineered PDE-related protein from (e).

71. The method according to any one of aspects 67 to 70, wherein said PDE-related protein is a PDE.

72. The method according to aspect 71, wherein said PDE is a PDE5-related protein.

73. The method according to aspect 72, wherein said PDE5-related protein is PDE5.

74. The method according to aspect 73, wherein said PDE5 is as defined in any one of aspects 5 to 7.

The compound of the present invention (i.e. a compound according to aspect 44 or aspect 45, or a PDE5 ligand or a PDE5 inhibitor compound according to aspect 49; hereinafter referred to as “the compound”) can be administered alone but, in human therapy, will generally be administered in admixture with a suitable pharmaceutical excipient, diluent or carrier selected with regard to the intended route of administration and standard pharmaceutical practice. Thus, the pharmaceutical compositions, pharmaceuticals and medicaments contemplated by the present invention may be formulated in various ways well-known to one of skill and administered by similarly well-known methods.

For example, the compound of the invention can be administered orally, buccally or sublingually in the form of tablets, capsules (including soft gel capsules), ovules, elixirs, solutions or suspensions, which may contain flavouring or colouring agents, for immediate-, delayed-, modified-, or controlled-release such as sustained-, dual-, or pulsatile delivery applications. The compound may also be administered via intracavernosal injection. The compound may also be administered via fast dispersing or fast dissolving dosage forms or in the form of a high-energy dispersion or as coated particles. Suitable pharmaceutical formulations of the compound may be in coated or un-coated form as desired.

Such tablets may contain excipients such as microcrystalline cellulose, lactose, sodium citrate, calcium carbonate, dibasic calcium phosphate, glycine and starch (preferably corn, potato or tapioca starch), disintegrants such as sodium starch glycollate, croscarmellose sodium and certain complex silicates, and granulation binders such as polyvinylpyrrolidone, hydroxypropylmethyl cellulose (HPMC), hydroxypropylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the compound may be combined with various sweetening or flavouring agents, colouring matter or dyes, with emulsifying and/or suspending agents and with diluents such as water, ethanol, propylene glycol and glycerin, and combinations thereof.

Modified release and pulsatile release dosage forms may contain excipients such as those detailed for immediate release dosage forms together with additional excipients that act as release rate modifiers, these being coated on and/or included in the body of the device. Release rate modifiers include, but are not exclusively limited to, hydroxypropylmethyl cellulose, methyl cellulose, sodium carboxymethylcellulose, ethyl cellulose, cellulose acetate, polyethylene oxide, Xanthan gum, Carbomer, ammonio methacrylate copolymer, hydrogenated castor oil, camauba wax, paraffin wax, cellulose acetate phthalate, hydroxypropylmethyl cellulose phthalate, methacrylic acid copolymer and mixtures thereof. Modified release and pulsatile release dosage forms may contain one or a combination of release rate modifying excipients. Release rate-modifying excipients maybe present both within the dosage form i.e. within the matrix, and/or on the dosage form i.e. upon the surface or coating.

Fast dispersing or dissolving dosage formulations (FDDFs) may contain the following ingredients: aspartame, acesulfame potassium, citric acid, croscarmellose sodium, crospovidone, diascorbic acid, ethyl acrylate, ethyl cellulose, gelatin, hydroxypropylmethyl cellulose, magnesium stearate, mannitol, methyl methacrylate, mint flavouring, polyethylene glycol, fumed silica, silicon dioxide, sodium starch glycolate, sodium stearyl fumarate, sorbitol, xylitol. The terms dispersing or dissolving as used herein to describe FDDFs are dependent upon the solubility of the drug substance used i.e. where the drug substance is insoluble a fast dispersing dosage form can be prepared and where the drug substance is soluble a fast dissolving dosage form can be prepared.

The compound can also be administered parenterally, for example, intracavemosally, intravenously, intra-arterially, intraperitoneally, intrathecally, intraventricularly, intraurethrally intrastemally, intracranially, intramuscularly or subcutaneously, or they may be administered by infusion or needleless injection techniques. For such parenteral administration they are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

For oral and parenteral administration to human patients, the daily dosage level of the compound will usually be from 10 to 500 mg (in single or divided doses).

Thus, for example, tablets or capsules of the compound may contain from 5 mg to 250 mg of active compound for administration singly or two or more at a time, as appropriate. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention. The skilled person will also appreciate that, in the treatment of certain conditions (including MED and FSD), the compound may be taken as a single dose on an “as required” basis (i.e. as needed or desired).

The compound can also be administered intranasally or by inhalation and are conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A™ or 1,1,1,2,3,3,3-heptafluoropropane (HFA 227EA™), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of a compound of the invention and a suitable powder base such as lactose or starch.

Aerosol or dry powder formulations are preferably arranged so that each metered dose or “puff” contains from 1 to 50 mg of a compound of the invention for delivery to the patient. The overall daily dose with an aerosol will be in the range of from 1 to 50 mg which may be administered in a single dose or, more usually, in divided doses throughout the day.

The compound may also be formulated for delivery via an atomiser. Formulations for atomiser devices may contain the following ingredients as solubilisers, emulsifiers or suspending agents: water, ethanol, glycerol, propylene glycol, low molecular weight polyethylene glycols, sodium chloride, fluorocarbons, polyethylene glycol ethers, sorbitan trioleate, oleic acid.

Alternatively, the compound can be administered in the form of a suppository or pessary, or they may be applied topically in the form of a gel, hydrogel, lotion, solution, cream, ointment or dusting powder. The compound may also be dermally administered. The compound may also be transdermally administered, for example, by the use of a skin patch. The compound may also be administered by the ocular, pulmonary or rectal routes.

For ophthalmic use, the compound can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, the compound may be formulated in an ointment such as petrolatum.

For application topically to the skin, the compound of the invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, it can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

The compound may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO-A-91/11172, WO-A-94/02518 and WO-A-98/55148.

Generally, in humans, oral administration of the compound is the preferred route, being the most convenient and, for example in MED, avoiding the well-known disadvantages associated with intracavernosal (i.c.) administration. A preferred oral dosing regimen in MED for a typical man is from 25 to 250 mg of compound when required. In circumstances where the recipient suffers from a swallowing disorder or from impairment of drug absorption after oral administration, the drug may be administered parenterally, sublingually or buccally.

For veterinary use, the compound, or a veterinarily acceptable salt thereof, or a veterinarily acceptable solvate or pro-drug thereof, is administered as a suitably acceptable formulation in accordance with normal veterinary practice and the veterinary surgeon will determine the dosing regimen and route of administration which will be most appropriate for a particular animal.

### DETAILED DESCRIPTION OF THE INVENTION

The term “apo” as used herein is taken to mean any protein (or named protein) that is detached from a/its ligand(s) and/or prosthetic group(s).

The term “active site” as used herein is taken to include any site (e.g. specific groups) within a molecule (and associated metal ions and/or hydration molecules) where specific activity is undergone. Such activity could include binding of a ligand to the site, catalysis of the molecule's substrates by the site, recognition of a ligand by the site, etc.

The term “buffer” as used herein is taken to include any solution containing a weak acid and a conjugate base of this acid (or, less commonly, a weak base and its conjugate acid). Thus, a “buffer” as used herein resists change in its pH level when an acid or a base is added to it, because the acid neutralises an added base (or, less commonly, the base neutralises an added acid).

The term “precipitant” as used herein is taken to include any substance that, when added to solutionm (usually of macromolecules), causes a precipitate to form or crystals to grow.

The term “complex” as used herein is taken to mean a protein with ligand(s) bound and may be formed before, during or after protein crystallisation.

The term “soaking” as used herein is taken to mean the addition of a solution containing a (usually) small molecule (e.g. inhibitor) to crystals of a protein to form a protein-ligand complex.

The term “co-crystallisation” as used herein is taken to mean crystallisation of a pre-formed protein/small molecule complex.

The terms “mutant”, “variant”, “homologue”, “analogue”, “derivative” or “fragment” in relation to the amino acid sequence of the crystal of the PDE5 of the present invention include any substitution of, variation of, modification of, replacement of, deletion of or addition of one (or more) amino acids from (or to) the sequence providing the resultant PDE5 is capable of being crystallised.

The terms “mutant”, “variant”, “homologue”, “analogue”, “derivative” or “fragment” in relation to the nucleotide sequence coding for the PDE5 of the crystal of the present invention include any substitution of, variation of, modification of, replacement of, deletion of or addition of one (or more) nucleic acid from (or to) the sequence providing the resultant nucleotide sequence codes for or is capable of coding for a PDE5 which is capable of being crystallised.

Typically, for the “mutant”, “variant”, “homologue”, “analogue”, “derivative” or “fragment” in relation to the amino acid sequence of the crystal of the PDE5 of the present invention, the types of amino acid substitutions that could be made should maintain the hydrophobicity/hydrophilicity of the amino acid sequence. Amino acid substitutions may be made, for example from 1, 2 or 3 to 10, 20 or 30 substitutions provided that the modified PDE5 retains the ability to be crystallised in accordance with present invention. Amino acid substitutions may include the use of non-naturally occurring analogues.

In relation to amino acid sequences, the term “variant” as used herein refers to additions, deletions or substitutions of amino acid residues comprised within the wild-type amino acid sequence or fragment thereof. Preferably, a variant in relation to the amino acid sequence of the crystal of the PDE5 of the present invention could include the deletion or substitution of the histidine (His/H) residue as shown emboldened and underlined in SEQ ID NO: 1 (HRGVNNSYIQRSEHPLAQLYCHSIME), which sequence is comprised within the PDE5 molecule of the crystal of the PDE5 of the present invention. Replacement of said histidine (H) residue is preferably by way of incorporating one or more amino acid residues (other than histidine), preferably wherein said amino acid residues are neutral or non-polar.

More preferably, a variant in relation to the amino acid sequence of the crystal of the PDE5 of the present invention includes the complete replacement of the loop region with a loop region (or other equivalent amino acid sequence e.g. sub-domain) from another protein, preferably a PDE, more preferably PDE4, most preferably PDE4b.

Alternatively, a variant in relation to the amino acid sequence of the crystal of the PDE5 of the present invention includes the deletion or substitution of the amino acid residues PLAQ (proline, leucine, alanine and glutamine) as emboldened and underlined in SEQ ID NO: 1 (HRGVNNSYIQRSEHPLAQLYCHSIME). Preferably, such substitution of amino acid residues utilises amino acids of similar charge to those substituted.

In relation to nucleotide sequences, the term “variant” as used herein refers to additions, deletions or substitutions of nucleotides comprised within the wild-type nucleotide sequence or fragment thereof.

The term “fragment” as used herein refers to any portion of the PDE5 as defined in the present invention provided the resultant PDE5 comprising said PDE5 portion is capable of being crystallised. Thus, the term “fragment” also includes PDE5, which comprises any portion of SEQ ID NOS: 1, 2, 3, 4, 5, or 6.

For example, a specific fragment of SEQ ID NO: 3 (full-length wild-type PDE5 sequence) according to the present invention could be SEQ ID NO: 2 (wild-type PDE5 catalytic domain). An example of a specific fragment of SEQ ID NO: 2 (wild-type PDE5 catalytic domain) according to the present invention could be SEQ ID NO: 1 (PDE5 “loop region”; HRGVNNSYIQRSEHPLAQLYCHSIME).

An example of a specific fragment of SEQ ID NO: 6 (full-length “loop-swapped” PDE5 sequence) according to the present invention could be SEQ ID NO: 5 (“loop-swapped” PDE5 catalytic domain). Moreover, an example of a specific fragment of SEQ ID NO: 5 (“loop-swapped” PDE5 catalytic domain) according to the present invention could be SEQ ID NO: 4 (PDE4 “loop region”; HPGVSNQFLINTNSELALMYNDESVLE).

The term “analogue” as used herein means a sequence similar to the amino acid sequence of the crystal of the PDE5 of the present invention or of any one of SEQ ID NOS: 1, 2, 3, 4, 5 or 6, but wherein non-detrimental (i.e. not detrimental to the PDE5's capability of being crystallised) amino acid substitutions or deletions have been made.

The term “derivative” as used herein in relation to the amino acid sequence of the crystal of the PDE5 of the present invention, or of any one of SEQ ID NOS: 1, 2, 3, 4, 5 or 6, includes chemical modification of PDE5. Illustrative of such modifications would be replacement of hydrogen by an alkyl, acyl, or amino group.

As used herein a “deletion” is defined as a change in either nucleotide or amino acid sequence in which one or more nucleotides or amino acid residues, respectively, are absent.

As used herein an “insertion” or “addition” is a change in a nucleotide or amino acid sequence, which has resulted in the addition of one or more nucleotides or amino acid residues, respectively, as compared to the naturally occurring PDE5.

As used herein “substitution” results from the replacement of one or more nucleotides or amino acids by different nucleotides or amino acids, respectively.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

The term “homologue” covers homology specifically with respect to structure and covers any structural PDE5 homologue that is capable of being crystallised.

With respect to homology of the amino acid sequences detailed herein, preferably there is at least 70%, more preferably at least 75%, more preferably at least 80%, yet more preferably at least 85%, even more preferably at least 90% homology to SEQ ID NOS: 1, 2, 3, 4, 5 or 6. More preferably there is at least 95%, and most preferably at least 98%, homology to SEQ ID NOS: 1, 2, 3, 4, 5 or 6.

With respect to homology of the nucleotide sequences coding for the amino acid sequences detailed herein, preferably there is at least 70%, more preferably at least 75%, more preferably at least 80%, yet more preferably at least 85%, even more preferably at least 90% homology to the nucleotide sequences which code for SEQ ID NOS: 1, 2, 3, 4, 5 or 6. More preferably there is at least 95%, and most preferably at least 98%, homology to the nucleotide sequences which code for SEQ ID NOS: 1, 2, 3, 4, 5 or 6.

The term “homologue” with respect to the nucleotide sequence of the PDE5 as defined in the present invention and the amino acid sequence of the PDE5 as defined in the present invention may be synonymous with allelic variations of the sequences.

In particular, the term “homology” as used herein may be equated with the term “identity”. Here, sequence homology with respect to, for example, the amino acid sequence of the crystal of the PDE5 of the present invention can be determined by a simple “eyeball” comparison (i.e. a strict comparison) of any one or more of the sequences with another sequence to see if that other sequence has at least 70% identity to the sequence(s). Relative sequence homology (i.e. sequence identity) can also be determined by commercially available computer programs that can calculate percentage (%) homology between two or more sequences. A typical example of such a computer program is CLUSTAL.

% homology may be calculated over contiguous sequences, i.e. one sequence is aligned with the other sequence and each amino acid in one sequence directly compared with the corresponding amino acid in the other sequence, one residue at a time. This is called an “ungapped” alignment. Typically, such ungapped alignments are performed only over a relatively short number of residues (for example less than 50 contiguous amino acids).

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software that can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for off-line and on-line searching (see Ausubel et al., 1999 ibid, pages 7-58 to 7-60). However, for some applications it is preferred to use the GCG Bestfit program.

Although the final % homology can be measured in terms of identity, in some cases, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). It is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

As indicated, for some applications, sequence homology (or identity) may be determined using any suitable homology algorithm, using for example default parameters. For a discussion of basic issues in similarity searching of sequence databases, see Altschul et al (1994) Nature Genetics 6:119-129. For some applications, the BLAST algorithm is employed, with parameters set to default values. The BLAST algorithm is described in detail at http://www.ncbi.nih.gov/BLAST/blast_help.html. Advantageously, “substantial homology” when assessed by BLAST equates to sequences which match with an EXPECT value of at least about 7, preferably at least about 9 and most preferably 10 or more. The default threshold for EXPECT in BLAST searching is usually 10.

Other computer program methods to determine identify and similarity between the two sequences include but are not limited to the GCG program package (Devereux et al 1984 Nucleic Acids Research 12: 387 and FASTA (Atschul et al 1990 J. Mol. Biol. 403-410).

The amino acid sequence of the PDE5 of the present invention present invention may be produced by expression of a nucleotide sequence coding for the same in a suitable expression system.

In addition, or in the alternative, the protein itself could be produced using chemical methods to synthesize a PDE5 amino acid sequence, in whole or in part. For example, peptides can be synthesized by solid phase techniques, cleaved from the resin, and purified by preparative high performance liquid chromatography (e.g. Creighton (1983) Proteins Structures and Molecular Principles, WH Freeman and Co., New York, N.Y., USA). The composition of the synthetic peptides may be confirmed by amino acid analysis or sequencing (e.g. the Edman degradation procedure).

Direct peptide synthesis can be performed using various solid-phase techniques (Roberge J Y et al, Science, Vol 269, 1995, pp. 202-204) and automated synthesis may be achieved, for example, using the ABI 431 A Peptide Synthesizer (Perkin Elmer, Boston, Mass., USA) in accordance with the instructions provided by the manufacturer. Additionally, the amino acid sequence of PDE5, or any part thereof, may be altered during direct synthesis and/or combined using chemical methods with a sequence from other subunits, or any part thereof, to produce a variant polypeptide.

The Structure of Wild-Type PDE5 Catalytic Domain Complexed with Sildenafil

A recombinant construct of the catalytic domain (E534-N875) of human PDE5 was expressed and the protein crystallised in complex with Sildenafil and its structure determined by multi-wavelength anomalous dispersion (Hendrickson et al. 1989).

At the time of structure solution this represented a novel fold, however, subsequently the structure of the catalytic domain of PDE4b was published (Xu et al. 2000). A topological comparison of the PDE5 catalytic domain with the structures in the Protein Data Bank (PDB) does not reveal significant additional homology with other known protein structures except for the PDE4 structure. Comparisons between the two structures have been made (FIG. 1 shows a sequence and secondary structural alignment of the two proteins). The structure and domain arrangement is virtually identical to that of PDE4, save that the second sub-domain highlighted in PDE4 is only partially present in the PDE5 structure, as detailed below.

The structure is composed of a single domain of 15 α helices arranged in a compact fold (FIG. 2). Within the overall domain, three sub-domains can also be defined. Helices 1 (H1 539-545) and 2 (H2 551-554) lie on the exterior of the protein and comprise the N-terminal region of the construct. These two helices do not overlay with the equivalent ones (H0, H1 and H2) in the PDE4 structure. This region is not well conserved across the PDE protein family. Helices 3 (H3 568-582), 4 (H4 584-588), 5 (H5 592-604), 6 (H6 615-631) and 7 (H7 640-652) form the first sub-domain of the protein and are contained within the core of the protein. There is no observable electron density for helices 8 and 9 based on the PDE4 nomenclature. Helix 10 (H10 684-694) is again on the exterior and forms the dimer interface within the structure. Helices 10 and 11 (H11 706-721) are the visible portion of the second sub-domain. Helices 12 (H12a 725-731, H12b 733-741), 13 (H13 749-765), 14 (H14 772-797), 15 (H145 813-824), 16 (H146 826-836) and 17 (H147 841-861) form the third sub-domain of the protein. It should be noted that in PDE5 helix H12 is not a contiguous helix as in PDE4 but is composed of two short helices with a kink in the middle and helix H15 is a contiguous helix in PDE5 but not in PDE4.

Dimer Assembly for Wild-Type PDE5-Sildenafil Complex:Catalytic Domain

There are four molecules present in the asymmetric unit, each molecule contains chain breaks and density is not visible for the C-terminal portion of the construct (see details below). The four molecules can be defined as two copies of a dimer. Molecule A (no electron density observed for residues: 534-536; 665-681; 863-875) is associated with molecule D (no electron density observed for residues: 534; 667-681; 865-875) and molecule B (no electron density observed for residues: 534-536; 667; 865-875) associated with molecule C (no electron density observed for residues: 534-53; 663-678; 863-875).

The molecules within the dimer are related by a two-fold rotation with the interface being formed by association of helix H10 from molecule A and D. Key to this dimer association is the presence of 2 zinc ions (one associated with each monomer). Residue His 683 from one molecule and His 684 and Asp 687 from the dimer partner co-ordinate each zinc ion. It is believed that the metal co-ordinated dimerisation is an artefact of crystallisation. The missing regions of structure in each molecule are believed to be due to the high flexibility of this part of the structure. Further it is believed that there is significant cleavage of the protein in this region which gives rise to much of the flexibility. This region corresponds to Helices H8 and H9 within the second sub-domain of the PDE4 structure.

Wild-Type PDE5-Sildenafil Complex: Active Site and Protein-Inhibitor Interactions

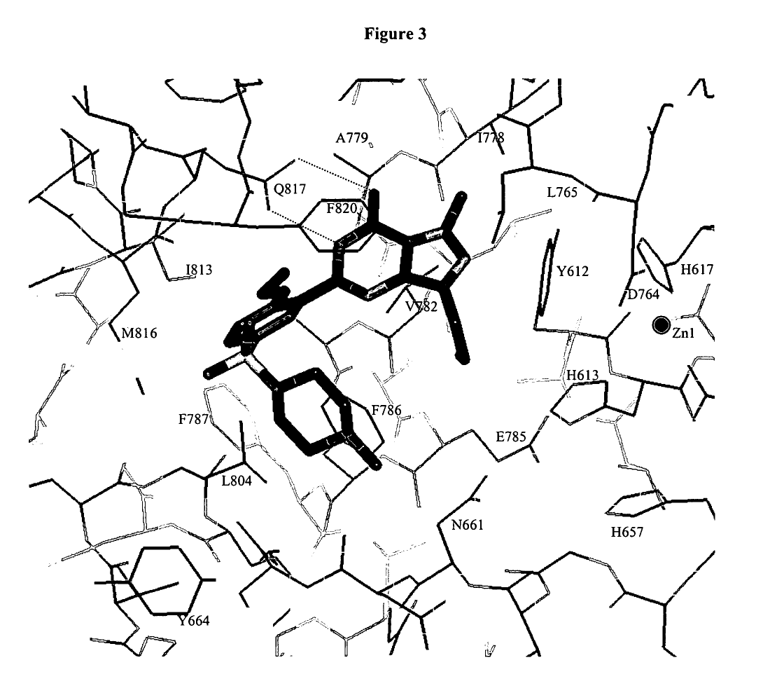

Each of the independently refined molecules in the structure contains one molecule of Sildenafil bound within the active site. The active site lies mainly within the third sub-domain of the protein and is bounded by helices H15, H14, the C-terminus of H13, and the C-terminus of H11 along with the loop region between H11 and H12a. The majority of the interactions between the inhibitor and the protein are hydrophobic in nature; with only two direct hydrogen bonds observed (FIG. 3). The first is between N17 of the purine ring of the inhibitor and Oε1 of Gln 817 (2.8 Å) and the second from the adjacent oxygen atom O16 of the inhibitor to Nε2 of the same residue Gln 817 (3.1 Å).

Carbon atom C12 of the inhibitor points into a small hydrophobic pocket formed by Leu 765, Ala 767 and Ile 768. These residues together with Phe 820 form a planar face to the binding site against which the purine ring of the inhibitor stacks. The opposite side of the purine packs against Val 782. The C5 propyl substituent form good van der Waals contacts with Val 782 and Phe 786 and Tyr 612. Phe 786 and Leu 804 form additional hydrophobic interactions with the phenyl moiety of the inhibitor. The O-alkyl moiety occupies a small pocket bounded by Ala 779, Phe 786, Ala 783, Val 782, Leu 804, Ile 813, Met 816 and Gln 817. The sulphonamide group points out towards the solvent whilst the piperazine ring is bounded by the extended residues 662-665, although whether the conformation of this part of the structure is unaffected by the chain break is questionable. There is no direct interaction between the inhibitor and the zinc ion found in the active site. The structure confirms the competitive nature of the mode of inhibition of Sildenafil by binding in the active site therefore blocking access for the cGMP substrate (which has also been modelled—data not included).

Wild Type PDE5-Sildenafil Complex: Metal Ions in the Active Site

Only one zinc atom is present in the active site of this structure. This can be clearly identified as a zinc atom since the phases used to determine the structure were obtained from a three-wavelength zinc MAD experiment. The anomalous signal observed clearly indicates the presence of a zinc ion. The co-ordination of the ion within the active site is also consistent with that expected for zinc. The metal is co-ordinated by His 653 (Nε2-Zn 2.0 Å), His 617 (Nε2-Zn 2.1 Å), Asp 764 (OD2-Zn 2.2 Å) and also Asp 654 (OD2-Zn 2.2 Å). These residues are completely conserved across the PDE gene family. There is no evidence of a second metal ion in the active site. A possible reason for the absence of any second metal ion in the active site is the sequestering of the metal ion (in this case a zinc, again confirmed by the anomalous signal) to form the dimer interface. Additionally there is the possibility that the residues likely to be involved in co-ordinating a second metal ion in the active site are not in the native conformation due to the proximity to the disordered region of the protein and the dimer interface.

Protein Engineering of PDE5*

Analysis of the catalytic domain protein by mass spectrometry and SDS Page gel electrophoresis (data not shown) shows that the protein is cleaved within the region not visible in the structure (residues 664-682). High concentrations of protease inhibitors provide some stabilisation of the protein, allowing the above structure to be determined.

An engineered form of the catalytic domain of PDE5 has been produced where the 657-682 region of PDE5 has been replaced by the same region in PDE4 producing a chimeric construct, (see FIG. 1 for sequence alignment of this region). The C-terminus of this construct is also truncated (C-term 858) compared to the wild-type structure (C-term 875). Hereafter this engineered construct will be referred to as ‘PDE5*’.

This engineered protein has been shown to be stable to degradation by mass spectrometry and SDS page gel electrophoresis (data not shown). The protein shows improved biophysical properties allowing an alternative purification protocol to be developed. The new protocol utilises binding to a blue sepharose column and specific elution with cGMP. The wild-type protein had been shown not to bind to this column probably due to the disorder of the structure around the protease cleavage site. This PDE5* protein was used to produce crystals with Sildenafil which diffract to higher resolution and have no disordered regions. The protein has also been used reproducibly to produce crystals with further inhibitors which routinely diffract to 1.8 Å resolution or higher, making it an improved protein for use in structure based drug design.

Structure of PDE5* Catalytic Domain with Sildenafil

The structure of the catalytic domain of PDE5* protein was determined by molecular replacement using the wild-type protein structure as a basis for the search model. This structure comprises 17 α helices and the overall fold is very similar to the wild-type structure with a number of important differences. The major difference in the structure is the presence of helices H8 and H9 composed of the swapped portion from PDE4, residues 657-682. These helices fold in an identical way to that observed in the PDE4 structure and complete the second sub-domain of the protein. The entire C-terminal region of this construct can also be built into the electron density leaving just three disordered residues at the N-terminus of this structure. This is likely to contribute to its enhanced properties for crystallisation. The PDE5* catalytic domain crystallises as a monomer with two molecules present in the asymmetric unit related by a translational shift. (PDE5* has also been crystallised with other inhibitors of PDE5 in space group P2₁ with one molecule in the asymmetric unit. The crystals have approximate unit cell dimensions a=56 b=77 c=83 Å α=γ=90° β=103°).

PDE5*: Active Site and Protein-Inhibitor Interactions

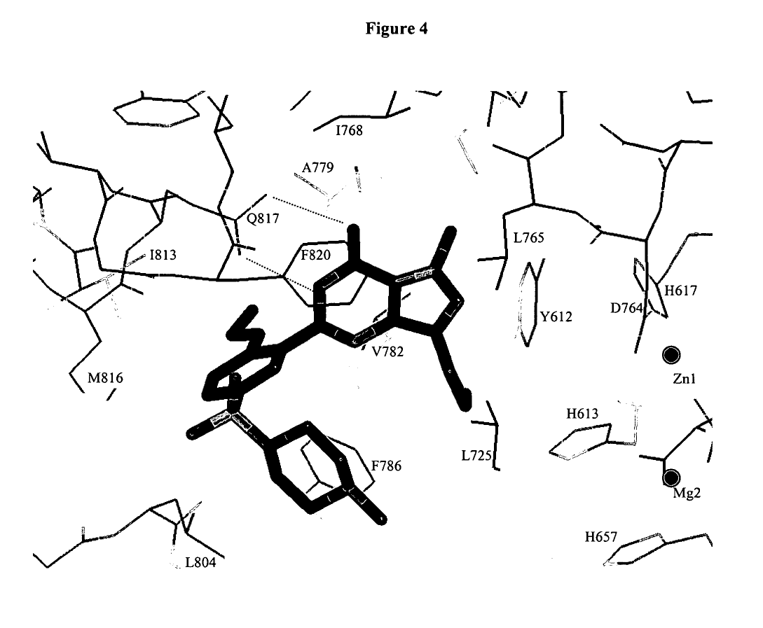

Each of the independently refined molecules again contains one molecule of Sildenafil in the active site. Sildenafil occupies the same region of the active site as observed in the wild-type structure forming the same mainly hydrophobic interactions with the protein (FIG. 4). The same two direct hydrogen bonds are formed between Gln 817 of the protein and inhibitor (Oε1-N17 2.8 Å and Nε2-O16 3.1 Å). The remainder of the inhibitor makes the same contacts with the sulphonylpiperazine again pointing out towards solvent. This is close to the engineered region of the protein but the piperazine ring forms no interactions with the ordered swapped region of the catalytic domain construct. This is an important factor when considering the use of this chimeric catalytic domain for drug design.

PDE5*: Metal Ions in the Active Site

Another notable difference in the structure of PDE5* compared with that of wild-type PDE5, is the presence of two metal ions in the active site. As observed in the wild-type complex there is no direct interaction between the inhibitor and the zinc ion found in the active site. There is also no direct interaction between Sildenafil and the second metal ion observed in this complex. This second metal ion is coordinated to Asp 764 (OD 1 2.15 Å) and to a water network that stabilises the metal environment. Due to the co-ordination geometry and the relative observed electron density, this second metal ion has been refined as a Mg²⁺ in accordance with a similar observation in the PDE4 structure solution. This structural arrangement is in accordance with the proposed mechanism, where an OH⁻ ion is derived from an H₂O molecule ionised by the presence of divalent metal atoms bound in the active site (Goldberg et al. 1980, Francis et al. 1994). The phosphodiester bond between the phosphorous and the oxygen atoms at the 3′ position of cGMP is then hydrolysed via OH⁻ nucleophilic attack.

The present invention will now be described, by way of example only, with reference to the accompanying Sequence Listing and Figures, in which:—

SEQ ID NO: 1 shows the amino acid sequence of the loop region from PDE5.

SEQ ID NO: 2 shows the amino acid sequence of the wild-type PDE5 catalytic domain.

SEQ ID NO: 3 shows the amino acid sequence of the full-length wild-type PDE5 sequence.

SEQ ID NO: 4 shows the amino acid sequence of the loop region of PDE4.

SEQ ID NO: 5 shows the amino acid sequence of the loop-swapped PDE5 catalytic domain ═PDE5*.

SEQ ID NO: 6 shows the amino acid sequence of full-length PDE5 sequence comprising PDE5*.

SEQ ID NOS: 7-14 are oligonucleotide primers.

FIG. 1 shows an alignment of PDE5 (upper sequence) and PDE4b (lower sequence) catalytic domains. Positions and numbering of helices from the structures are marked for each. Residues in bold show a sequence alignment for the engineered region. The sequence from PDE4 has been used to replace the corresponding region in PDE5. This results in a residue insertion in this region. Underlining highlight C-terminal region absent in PDE5*.

FIG. 2 shows a ribbon representation of the overall fold of proteins showing secondary structure elements. The inhibitor is shown in an all atom stick representation and the metal ions as spheres. (A)=PDE4b, (B)=wild-type PDE5+Sildenafil, (C)=“loop-swapped” PDE5 (PDE5*)+Sildenafil. Helices are numbered using PDE4 structure as reference. Helices H0-H7 form sub-domain 1, helices H8-H11 form sub-domain 2, and helices H12-H16 form sub-domain 3.

FIG. 3 shows a view of compound Sildenafil bound to wild-type PDE5.

FIG. 4 shows a view of compound Sildenafil bound to “loop-swapped” PDE5 (PDE5*).

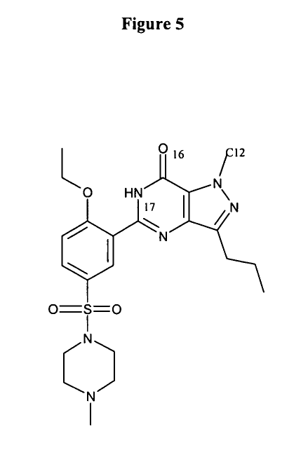

FIG. 5 shows the chemical structure of the inhibitor Sildenafil.

### EXPERIMENTAL SECTION

### Example 1

### Construction and Expression of PDE5 Wild-Type Catalytic Domain (E534-N875)

Oligonucleotide primers were designed from the sequence of human PDE5 (Accession number=AB001635). DNA fragments were generated by PCR amplification from a full-length PDE5 clone. The following oligonucleotides were used:

PDE5 5′Untagged Oligo:

(SEQ ID NO: 7)

CGTGAATTCATGGAGGAAGAAACAAGAGAGCTAC
PDE5 3′Long Oligo:

(SEQ ID NO: 8)

CGTTCTAGACTATCAGTTCCGCTTGGCCTGGCCGCTTTCCCC

The PCR reaction was carried out for 30 cycles in a total volume of 50 μl in a solution containing 1.5 mM MgCl₂, 200 μM dNTPs, 50 pmol of each primer and 2.5 units of Expand DNA polymerase (Roche, East Sussex, UK). Each cycle was 94° C., 1 min, 50° C., 1 min and 72° C., 2 mins.

The final amplified DNA fragments for both constructs were separated on a 1% agarose gel and purified using a QIAquick gel extraction kit (Qiagen, West Sussex, UK). The fragment was then digested using EcoRI and XbaI, and ligated into pFastbac1 EcoRI/XbaI-digested vector (Life Technologies, Paisley, UK). The ligation was carried out at 12° C. for 16 hours. The ligation mix was then electroporated into E. coli (TOP 10) (Invitrogen, Gronigen, The Netherlands).

Clones containing the desired insert were selected by using 2YT plates containing 100 μg/ml ampicillin and checked using endonuclease digestion for presence of correct size insert. DNA sequence analysis was carried out by Lark (Saffron Waldon, UK).

Recombinant bacmid DNA was produced by transforming E. coli DH10BAC™ with pFastbacl::PDE5 catalytic domain (534-875) plasmid DNA. This was carried out according to the method shown in the Bac to Bac™ baculovirus expression manual (Life Technologies, Paisley, UK). PCR analysis was used to verify successful transposition to the bacmid using pUC/M13 amplification primers (Invitrogen, Gronigen, The Netherlands).

Generation of primary baculovirus stocks was carried out by transfection using Sf-9 insect cells. Bacmid DNA containing the correct insert was mixed with CELLFECTIN™ transfection reagent (Life Technologies, Paisley, UK) and added to a monolayer of Sf-9 insect cells using SF-900II serum free medium (Invitrogen, Gronigen, The Netherlands). Following 72 hours incubation at 27° C. the supernatant was harvested as the initial baculovirus stock. This stock was amplified by adding the initial virus stock into a suspension of Sf-9 insect cells at 1×10⁶ cells/ml in 1 litre Erlenmeyer flasks (Coming Life Sciences, New York, USA), at an agitation of 125 rpm at 27° C. After 6 days post-infection the supernatant was harvested by centrifugation and stored at 4° C. as the working virus stock. The titre of this working stock was determined by conventional plaque assay analysis as in the Bac to Bac™ baculovirus expression manual (Invitrogen, Gronigen, The Netherlands).

Protein expression was optimised in Erlenmeyer flask cultures using Sf-9 and T. ni High5 insect cell cultures looking at different multiplicity's of infection (MOI) and harvest times, the optimal conditions found were then scaled up into fermenters.

The fermenters used were autoclavable Applikon 3 litre stirred vessels controlled using Applikon 1030 biocontrollers. Inoculum of T. ni High5 cells was initially prepared from shake flask cultures. The fermenter was inoculated with 5×10⁵ cells/ml, with an initial working volume of 1.8 litres made up in Excel 405 serum free medium (JRH Biosciences, Kansas, USA). Temperature was controlled at 27° C., dissolved oxygen concentration controlled at 60% and pH was measured but not controlled. Oxygen concentration was controlled throughout. Agitation was set at 150 rpm with a double impeller system of marine impeller within the culture and Rushton impeller at the liquid/headspace interface. Aeration was continuous to the headspace at 0.5 l/min.

When the viable cell density reached 2×10⁶ cells/ml the culture was infected using an MOI of 1 from the titred baculovirus working stock. Harvest time for the culture was 48 hours post-infection. This was achieved by centrifugation at 2000 g for 15 mins; the insect cell pellet was then stored at −80° C. prior to purification.

### Example 2

### Construction and Expression of His6-Tagged PDE5 Wild-Type Catalytic Domain (E534-N875)

Oligonucleotide primers were designed from the sequence of human PDE5 (═PDE5A1 isoform; Accession number=AB001635). DNA fragments were generated by PCR amplification from a full-length PDE5 clone. The following oligonucleotides were used:

PDE5 5′His6 Oligo

(SEQ ID NO: 9)

CGTGAATTCATGCATCATCATCATCATCATCTTCTGGTTCCG
CGTGGATCTGCGCCCGAGGAAGAAACAAGAGAGCTAC
PDE5 3′Long Oligo

(SEQ ID NO: 8)

CGTTCTAGACTATCAGTTCCGCTTGGCCTGGCCGCTTTCCCC

The PCR reaction was carried out for 30 cycles in a total volume of 50 μl in a solution containing 1.5 mM MgCl₂, 200 μM dNTPs, 50 pmol of each primer and 2.5 units of Expand DNA polymerase (Roche, East Sussex, UK). Each cycle was 94° C., 1 min, 50° C., 1 min and 72° C., 2 mins.

The final amplified DNA fragments for both constructs were separated on a 1% agarose gel and purified using a QIAquick gel extraction kit (Qiagen, West Sussex, UK). The fragment was then digested using EcoRI and XbaI, and ligated into pfastbac1 EcoRI/XbaI-digested vector (Life Technologies, Paisley, UK). The ligation was carried out at 12° C. for 16 hours. The ligation mix was then electroporated into E. coli (TOP 10) (Invitrogen, Gronigen, The Netherlands).

Clones containing the desired insert were selected by using 2YT plates containing 100 μg/ml ampicillin and checked using endonuclease digestion for presence of correct size insert. DNA sequence analysis was carried out by Lark (Saffron Waldon, UK).

Methods to generate the recombinant baculovirus were as those for wild-type PDE5 catalytic domain (see EXAMPLE 1).

Expression optimisation again showed T. ni High5 insect cells to give the best expression. Therefore baculovirus expression in fermenters was carried out using the same procedures as for the previous construct.

### Example 3

### Construction and Expression of PDE5* (E534-E858) in Baculovirus

The PDE5* construct was produced by using overlap extension PCR where the following oligonucleotides were used:

(SEQ ID NO: 7)

A: CGTGAATTCATGGAGGAAGAAACAAGAGAGCTAC

(SEQ ID NO: 10)

B: CAAAGAAAGTTCTGAATTTGTGTTGATGAGAAACTGATTGGAGACTC
CAGGATGATCCAAATCGTGGCTTAG

(SEQ ID NO: 11)

C: ATCAACACAAATTCAGAACTTGCTTTGATGTATAATGATGAATCTGT
GTTGGAACACCATCATTTTGACCAG

(SEQ ID NO: 12)

D: CGTTCTAGACTATCATTCTGCAAGGGCCTGCCATTTCTG

Initial DNA fragments were generated using oligonucleotides A+B and C+D with the same template DNA as for the wild-type PDE5 catalytic domain construct. The PCR reaction was carried out for 30 cycles in a total volume of 50 μl in a solution containing 1.5 mM MgCl₂, 200 μM dNTPs, 50 pmol of each primer and 2 units of Expand DNA polymerase (Roche, East Sussex, UK). Each cycle was 94° C., 1 min, 50° C., 2 min, and 72° C., 3 min. In the second round of PCR, DNA products from PCR A+B and C+D were used as template DNA with the oligonucleotides A+D used to amplify the full-length construct. The PCR conditions were the same as the initial PCR reaction. This generates a construct with the PDE4 swapped region and a C-terminal truncation (C-term 858) as compared to PDE5 catalytic domain (C-term 875).

Methods to generate the recombinant baculovirus were as those for wild-type PDE5 catalytic domain (see EXAMPLE 1).

Expression optimisation again showed T. ni High5 insect cells to give the best expression. Therefore baculovirus expression in fermenters was carried out using the same procedures as for the previous construct.

### Example 4

### Construction and Expression of PDE5* (E534-E858) in E. coli

The PDE5* construct in E. coli was produced by using PCR where the following oligonucleotides were used and the template DNA being pFastbac1::PDE5* plasmid DNA (sequence verified), produced in EXAMPLE 3.

(SEQ ID NO: 13)

E: CGTCATATGGAGGAAGAAACAAGAGAGCTAC

(SEQ ID NO: 14)

F: CGTCTCGAGCTATCATTCTGCAAGGGCCTGCCATTTCTG

The PCR reaction was carried out for 30 cycles in a total volume of 50 μl in a solution containing 1.5 mM MgCl₂, 200 μM dNTPs, 50 pmol of each primer and 2.5 units of Expand DNA polymerase (Roche, East Sussex, UK). Each cycle was 94° C., 1 min, 50° C., 1 min and 72° C., 2 mins.

The final amplified DNA fragment was separated on a 1% agarose gel and purified using a QIAquick gel extraction kit (Qiagen, West Sussex, UK). The fragments were then digested using Nde1 and Xho1, and ligated into pET21 C (Novagen, Nottingham, UK) Nde1/Xho1-digested vector. The ligation was carried out at 12° C. for 16 hours. The ligation mix was then electroporated into E. coli (TOP 10) (Invitrogen, Gronigen, The Netherlands).

Clones containing the desired insert were selected by using 2YT plates containing 100 μg/ml ampicillin. Plasmid DNA was also checked using endonuclease digestion for presence of correct size insert. DNA sequence analysis was carried out by Lark (Saffron Waldon, UK).

The correctly sequenced plasmid DNA was then electroporated into E. coli BL21 (DE3) (Novagen, Nottingham, UK) for expression. Expression was carried out in 7 litre Applikon fermenters using 5 litre 2YT broth containing 100 μg/ml carbenicillin as the medium. Agitation was set at 1000 rpm using a double rushton impeller assembly and aeration to the sparger at 2 litres/min. The fermenter was inoculated with an overnight shake flask culture grown at 37° C. and 200 rpm, the inoculation density was 1% vol/vol. The fermentation was pH controlled at 7.2 using 20% vol/vol NH₄OH solution and temperature initially set to 37° C. When the OD₆₀₀ₙₘ reached 1.5 the temperature set-point was reduced to 25° C. and then the culture was induced with IPTG at a final concentration of 1 mM. The fermentation was then harvested 4 hours post-induction by batch centrifugation (8,000 rpm for 10 minutes). The final pellet was then frozen (−80° C.) to await subsequent purification.

### Example 5

### Purification of Wild-Type PDE5 Catalytic Domain

Pellet from the fermentation was resuspended into 10 mls lysis buffer per gram wet cell weight and mechanically broken using a continuous cell disrupter (Constant Systems, Warwickshire, UK) at a pressure of 20 kpsi. The lysis buffer consisted of 50 mM Tris HCl (pH 7.2), 100 mM NaCl, 1 mM DL-dithiothreitol (DTT) containing EDTA-free complete protease inhibitor cocktail tablets (Roche, East Sussex, UK) and 10 μM epoxysuccinyl-1-leucylamido-(4-guanidino)butane (E-64) (Sigma, Dorset, UK; Catalogue No. E-3132). The lysate was chilled and centrifuged at 14000 g for 45 min to remove cell debris. All purifications were subsequently carried out using an Akta Explorer purification system (Amersham Pharmacia, Buckinghamshire, UK). The supernatant was applied to a 50 ml Q-sepharose fast-flow column (Amersham Pharmacia, Buckinghamshire, UK) at 5 ml/in the flow-through was directly applied to a 20 ml Nickel chelate column (Amersham Pharmacia, Buckinghamshire, UK) previously charged with 0.1 M NiSO₄. The Nickel chelate column was washed with 5 column volumes of lysis buffer. The column was then step-eluted with lysis buffer containing 50 mM imidazole. This elution fraction was directly applied to a 2 litre G-25 superfine desalting column (Amersham Pharmacia, Buckinghamshire, UK) equilibrated in SP-sepharose buffer A (25 mM Bis-Tris (pH 6.5), 50 mM NaCl, 1 mM DTT and 2 μM E-64). The protein was eluted in this buffer at 50 ml/in. The eluted fraction was then loaded onto a 20 ml SP-sepharose high-performance column (Amersham Pharmacia, Buckinghamshire, UK) at a flow-rate of 5 mls/min. The flow-through was collected and dialysed overnight at 4° C. in Heparin buffer A (25 mM Bis-Tris (pH 6.5), 1 mM DTT and 2 μM E-64). Dialysis volume equalled 50 times the protein sample volume and the dialysis tubing used was 10 kDa Snakeskin™ (Pierce, Cheshire, UK).

The dialysed sample was then loaded onto a 20 ml Heparin sepharose column (Amersham Pharmacia, Buckinghamshire, UK), equilibrated in Heparin buffer A. The column was eluted using a 10 column volume linear gradient with Heparin buffer A containing 300 mM NaCl at a flow-rate of 3 ml/min. Fractions containing PDE5 catalytic domain (534-875) were pooled and concentrated to 2 mg/ml using centrifugal protein concentrators (Vivascience, Gloucestershire, UK) and loaded at 1.5 m/min onto a Superdex-200 prep grade 26/60 column pre-equilibrated with 50 mM Bis-Tris (pH 6.8), 500 mM NaCl, 1 mM DTT and 2 μM E-64. The eluted fractions were analysed on Tris-glycine SDS PAGE gels.

### Example 6

### Purification of Wild-Type PDE5 Catalytic Domain (for apo Crystallisation)

Pellet from the fermentation was resuspended into 5 mls lysis buffer per gram wet cell weight and mechanically broken using a continuous cell disrupter (Constant Systems, Warwickshire, UK) at a pressure of 20 kpsi. The lysis buffer consisted of 50 mM Bis-Tris (pH 6.8), 10 mM imidazole, 10% glycerol, 50 mM sodium chloride and 3 mM β-mercaptoethanol (β-ME) containing EDTA-free complete protease inhibitor cocktail tablets (Roche, East Sussex, UK). The lysate was chilled and centrifuged at 13000 g for 30 min to remove cell debris then passed through a 0.2 μm filter. All purifications were subsequently carried out using FPLC purification system (Amersham Pharmacia, Buckinghamshire, UK). The supernatant was applied to a 20 ml Nickel chelate column (Amersham Pharmacia, Buckinghamshire, UK) previously charged with 0.1 M NiSO₄. The Nickel chelate column was washed with 10 column volumes of buffer A (Lysis buffer with Complete protease inhibitor tablets omitted) followed by 10 column volumes of buffer A containing 50 mM imidazole. The column was then eluted with a gradient of 100-500 mM imidazole in buffer A. These eluted fractions were analysed using Tris-glycine SDS-PAGE and stored overnight at 4° C. Fractions containing PDE5 catalytic domain were concentrated to 1.5 mg/ml using Centriprep 10 kDa Molecular weight cut-off centrifugal concentrators (Amicon Bioseparations, Maine, USA) at 3,000 rpm, 4° C. Half of the concentrated fraction was then loaded onto a 320 ml Sephacryl S300HR column (Amersham Pharmacia, Buckinghamshire, UK) pre-equilibrated in 50 mM Bis-Tris pH 6.8, 10% glycerol, 50 mM NaCl and 1 mM DL-dithiothreitol (DTT) at a flow-rate of 2 ml/min. The eluted fractions were analysed using Tris-glycine SDS-PAGE and those containing PDE5 catalytic domain were stored at −80° C.

### Example 7

### Purification of PDE5* Catalytic Domain

Pellet from both the E. coli and baculovirus fermentation was resuspended into 10 mls lysis buffer per gram wet cell weight and mechanically broken using a continuous cell disrupter (Constant Systems, Warwickshire, UK) at a pressure of 20 kpsi. The lysis buffer consisted of 50 mM Tris HCl (pH 7.5), 100 mM NaCl, 1 mM DTT containing EDTA-free protease inhibitor cocktail tablets (Roche, East Sussex, UK) and 10 μM E-64. The lysate was chilled and centrifuged at 14000 g for 45 min to remove cell debris. All purifications were subsequently carried out using an Akta Explorer purification system (Amersham Pharmacia, Buckinghamshire, UK). The supernatant was applied to a 50 ml Q-sepharose fast-flow column (Amersham Pharmacia, Buckinghamshire, UK) at 5 ml/min with the flow-through collected. The flow-through sample was then applied at 50 ml/min to a 2 litre G-25 superfine desalting column (Amersham Pharmacia, Buckinghamshire, UK) pre-equilibrated in Blue sepharose buffer A (50 mM Bis-Tris (pH 6.4), 50 mM NaCl, 2 mM EDTA, 2 mM EGTA and 1 mM DTT). The protein fraction was eluted in Blue sepharose buffer A.

The next column step was carried out in series, loading the sample initially onto a 20 ml SP-sepharose high performance column (Amersham Pharmacia, Buckinghamshire, UK) then flow-through from this directly onto a 10 ml Blue sepharose fast-flow column (Amersham Pharmacia, Buckinghamshire, UK) at a flow-rate of 2 ml/min. Once loading was complete, the SP-sepharose column was taken out of line and the Blue sepharose column washed with 5 column volumes of Blue sepharose buffer A. The column was washed with Blue sepharose buffer A containing 1 M NaCl until the absorbance 280 nm reached baseline and then washed with 5 column volumes of Blue sepharose buffer A. PDE5* protein was step-eluted using Blue sepharose buffer containing 20 mM cGMP (Na-salt) (Sigma, Dorset, UK). Fractions were assayed on Tris-glycine SDS gels (Invitrogen, Gronigen, The Netherlands) and pooled accordingly. These fractions were concentrated to 2.5 mg/ml using centrifugal concentrators (Vivascience, Gloucestershire, UK) and loaded at 1.5 m/min onto a Superdex-200 prep grade 26/60 column pre-equilibrated with 50 mM Bis-Tris (pH 6.8), 500 mM NaCl, 1 mM DTT and 2 μM E-64. The eluted fractions were analysed on Tris-glycine SDS PAGE gels.

### Example 8

### Crystallisation of Wild-Type His6-Tagged PDE5 Catalytic Domain (apo)

PDE5 fractions from the final gel filtration column were thawed from −80° C. and protein concentration measured. The solution was concentrated to 5.8 mg/ml using a Centriprep 10 kDa Molecular weight cut-off centrifugal concentrator (Amicon Bioseparations, Maine, USA) at 3,000 rpm, 20° C. then transferred to a Centricon 10 kDa Molecular weight cut-off centrifugal concentrator (Amicon Bioseparations, Maine, USA) and concentrated to 12.8 mg/ml at 4,000 rpm, 20° C. The protein solution was diluted to 10 mg/ml using ultrafiltrate from the final stage of concentration and frozen at −80° C. Prior to crystallisation, the protein solution was thawed and centrifuged for 2 min at 14,000 rpm in an Eppendorf centrifuge.

Hanging drop vapour diffusion crystallisation trials were set up at 20° C. Drops comprised of 2 μl reservoir buffer mixed with 2 μl protein solution were suspended on siliconised cover slips over 950 μl reservoir solutions containing 50 mM HEPES pH 7.6, 1.1M monobasic sodium phosphate and 1.1M monobasic potassium phosphate (all from Sigma, Dorset, UK). Both crystallisation plates and reservoir solutions were chilled to 4° C. before set up. Completed plates were placed in a 4° C. cold room. Rod shaped crystals, up to 400 μm in largest dimension, grew from precipitate after 1-2 weeks. Crystals were transferred gradually at 4° C., via solutions of increasing glycerol concentration, to a solution containing 0.1 M HEPES pH 7.6, 2.3M monobasic sodium phosphate and 20% glycerol as a cryoprotectant. Samples were then flash-frozen prior to X-ray data collection.

### Example 9

### Crystallisation of Wild-Type PDE5 Catalytic Domain with Sildenafil

The PDE5 fractions from the final gel filtration column were pooled (total volume of 25 ml) and the protein concentration was assayed (0.2 mg/ml). The protein solution was supplemented with 10 μM E-64 and 1 mg/ml leupeptin (Sigma, Dorset, UK). The solution was concentrated to 3 mg/ml using a Centriprep 10 kDa Molecular weight cut-off centrifugal concentrator (Amicon Bioseparations, Maine, USA) at 3,000 rpm, 4° C. A three-fold molar equivalent of Sildenafil (10 mg/ml aqueous stock solution) was added to the protein solution, which was then further concentrated to 8 mg/ml. A further one-molar equivalent of Sildenafil was added to this solution, which was concentrated to 10 mg/ml. Prior to crystallisation, the protein solution was centrifuged for 5 min at 14,000 rpm in an Eppendorf centrifuge.

Hanging drop vapour diffusion crystallisation trials were set up at 20° C. Drops comprised of 2 μl reservoir buffer mixed with 2 μl protein solution were suspended on siliconised cover slips over 900 μl reservoir solutions containing 0.1 M Tris pH 8.0, 50 mM ammonium phosphate, pH 7.0, 16-26% PEG2KMME (all from Sigma, Dorset, UK). Block shaped crystals, up to 300 μm in largest dimension, grew from precipitate after 2-5 days. Crystals were transferred to a solution containing 0.1 M Tris, pH 8.0, 250 mM NaCl, 10% glycerol and 12-22% PEG2KMME as a cryoprotectant. Samples were then flash-frozen prior to X-ray data collection.

### Example 10

### Crystallisation of PDE5*

The PDE5* fractions from the final gel filtration column were pooled (total volume of 25 mls) and the protein concentration was assayed (0.2 mg/ml). The protein solution was supplemented with 10 μM E-64 and 1 mg/ml leupeptin (Sigma, Dorset, UK). The solution was concentrated to 10 mg/ml using a Centriprep 10 kDa Molecular weight cut-off centrifugal concentrator (Amicon Bioseparations, Maine, USA) at 3,000 rpm, 4° C. Prior to crystallisation, the protein solution was centrifuged for 5 min at 14,000 rpm in an Eppendorf centrifuge.

Hanging drop vapour diffusion crystallisation trials were set up at 4° C. Drops comprised of 2 μl reservoir buffer mixed with 2 μl protein solution were suspended on cover slips over 900 μl-reservoir solutions containing 0.2M Sodium Acetate, 0.1 M Tris hydrochloride pH 8.5, 30% w/v Polyethylene Glycol 8000 (The solution is component number 22 in the Crystal Screen® from Hampton Research, California, USA.) Plate-shaped crystals, up to 700 μm in largest dimension, grew after 1-2 days. Crystals were transferred to a solution containing 0.16M Sodium Acetate, 80 mM Tris hydrochloride pH 8.5, 24% w/v Polyethylene Glycol 8000 and 10% glycerol and then frozen during X-ray data collection.

### Example 11

### Crystallisation of PDE5* with Sildenafil

Purified PDE5* protein was supplemented with 10 μM E-64 and 1 mg/ml leupeptin (Sigma, Dorset, UK).

Following the exact method described above for wild-type PDE5 catalytic domain, a complex of PDE5* with Sildenafil was made, and concentrated to a final protein concentration of 10 mg/ml.

Hanging drop vapour diffusion crystallisation trials were set up at 4° C. Drops comprised of 2 μl reservoir buffer mixed with 2 μl protein solution were suspended on siliconised cover slips over 900 μl reservoir solutions containing 0.1 M Tris pH 7.4, 50 mM ammonium phosphate, pH 7.5, 30-24% PEG2KMME (Sigma, Dorset, UK). Thin plate crystals grew after 2-5 days, the largest of these, up to 600 μm in the largest dimension, from the 28% PEG2KMME conditions.

Crystals were transferred to a solution containing 0.1 M Tris pH 7.4, 250 mM NaCl, 10% glycerol and 26-20% PEG2KMME as a cryoprotectant. Samples were then flash-frozen prior to X-ray data collection.

### Example 12

### Data Collection, Structure Determination and Refinement of Wild-Type PDE5

The structure of recombinant human PDE5 was solved by multiple wavelength anomalous dispersion (MAD) using four wavelengths at the zinc L^{lll} edge.

Native X-ray diffraction data were collected with an MAR CCD at station BM14 at the ESRF, Grenoble, France. All data were processed using the HKL package (Otwinowski & Minor, 1997). Data collection statistics are summarised in Table 1a.

The crystals belong to space group P6₂ with unit cell dimensions a=94.921 Å, b=94.921 Å, c=81.850 Å, a=β=90°γ=120°. They contain 1 molecule per asymmetric unit (M_{w}=39,654.71 Da) and have a calculated solvent content of 43.23% (V_{M}=2.18; Matthews, 1968).

Anomalous heavy atom sites were located using SOLVE (Terwilliger & Berendzen, 1997). Refinement of the heavy atom parameters and phase calculation was performed with SHARP (de La Fortelle & Bricogne, 1997). Phases were improved by 100 cycles of solvent flattening with SOLOMON (Abrahams & Leslie, 1996). The resulting map was of good quality and used to trace about 70% of the structure using QUANTA (Quanta98, 1998, version 98.1111; Molecular Simulations Inc., San Diego, Calif. 92121-3752, USA).

The model was refined against a set of native structure factors (F_{P}-calc) derived with SHARP from a combination of experimental native (F_{P}) and derivative (F_{PH}) structure factors. Refinement was carried out in the resolution range 30-2.5 Å using XPLOR (Brunger et al., 1998). Partial structure factors from a flat bulk-solvent model and anisotropic B-factor correction were supplied throughout the refinement. The R-factor for the current model is 0.260 (free R-factor, 5% of data, 0.319) for all data in the resolution range 30-2.5 Å. The refinement statistics are summarised in Table 2a.

The current model contains 296 out of 342 amino acid residues calculated on the basis of the construct and is well defined in most regions of the polypeptide chain. No interpretable electron density is observed for residues: 534, 657-673, 790-804 and 863-875.

Analysis of the structure using PROCHECK (Laskowski, et al., 1993) shows 12 residues from the four molecules in the asymmetric unit are in disallowed regions.

### Example 13

### Data Collection, Structure Determination and Refinement of Wild-Type PDE5 with Sildenafil

The structure of recombinant human PDE5 was solved by multiple wavelength anomalous dispersion (MAD) using three wavelengths at the zinc L^{lll} edge.

Native X-ray diffraction data were collected with an MAR CCD at station BM14 at the ESRF, Grenoble, France. All data were processed using the HKL package (Otwinowski & Minor, 1997). Data collection statistics are summarised in Table 1b.

The crystals belong to space group P2₁2₁2₁ with unit cell dimensions a=94.179 Å, b=103.645 Å, c=141.942 Å, α=β=γ=90°. They contain 4 molecules per asymmetric unit (M_{w}=39,654.71 Da) and have a calculated solvent content of 43.23% (V_{M}=2.18; Matthews, 1968).

Anomalous heavy atom sites were located using SOLVE (Terwilliger & Berendzen, 1997) and confirmed with SnB (Smith et al., 1998). Refinement of the heavy atom parameters and phase calculation was performed with SHARP (de La Fortelle & Bricogne, 1997). Phases were improved by 100 cycles of solvent flattening with SOLOMON (Abrahams & Leslie, 1996). The resulting map was of good quality and used to trace about 80% of the structure using QUANTA (Quanta98, 1998, version 98.1111; Molecular Simulations Inc., San Diego, Calif. 92121-3752, USA).

The model was refined against a set of native structure factors (F_{P}-calc) derived with SHARP from a combination of experimental native (F_{P}) and derivative (F_{PH}) structure factors. Refinement was carried out in the resolution range 30-2.2 Å using CNX (Brunger et al., 1998) with the “mlhl” maximum likelihood target function. Partial structure factors from a flat bulk-solvent model and anisotropic B-factor correction were supplied throughout the refinement. The R-factor for the current model is 0.235 (free R-factor, 5% of data, 0.28) for all data in the resolution range 30-2.2 Å. The refinement statistics are summarised in Table 2b.

The current model contains 1261 out of 1364 amino acid residues calculated on the basis of the construct and is well defined in most regions of the polypeptide chain. No interpretable electron density is observed for residues: Molecule A 534-536, 665-681 and 863-875; molecule D 534, 667-681 and 865-875; molecule B 534-536, 667 and 865-875 and molecule C 534-536, 663-678 and 863-875.

Analysis of the structure using PROCHECK (Laskowski, et al., 1993) shows only four residues from the four molecules in the asymmetric unit are in disallowed regions.

### Example 14

### Data Collection, Structure determination and Refinement of PDE5*

The structure of the baculovirus engineered PDE5* was solved by molecular replacement (MR) using the PDE5* coordinates obtained from the complex with Sildenafil (see EXAMPLE 15).

X-ray diffraction data were collected with an RaxisIV image plate detector on an in-house RU200HB rotating anode (Rigaku), with Blue Osmic mirrors (MSC). All data were processed using the HKL package (Otwinowski & Minor, 1997). Data collection statistics are summarised in Table 1a.

The crystals belong to the monoclinic space group P2₁, with unit cell dimensions a=54.983 Å, b=77.153 Å, c=80.660 Å, α=γ=90° β=101.311°. They contain 2 molecules per asymmetric unit (M_{w}=37,562 Da) and have a calculated solvent content of 45.1% (V_{M}=2.26; Matthews, 1968).

Molecular replacement was performed using AMORE (CCP4). The resulting map was of good quality and the structure was refitted using QUANTA. Refinement was carried out in the resolution range 30-1.6 Å using CNX (Brünger et al., 1998) with the “mlf” maximum likelihood target function. Partial structure factors from a flat bulk-solvent model and anisotropic B-factor correction were supplied throughout the refinement. The R-factor for the current model is 0.301 (free R-factor, 5% of data, 0.326) for all data in the resolution range 30-1.6 Å. The refinement statistics are summarised in Table 2a.

The current model contains 323 residues per molecule, 537-858 (residue Glu 681A has been numbered to maintain PDE5 numbering scheme). No interpretable electron density is observed for residues: 534, 535 and 536 in molecules A or B. Analysis of the structure using PROCHECK (Laskowski, et al., 1993) shows only two residues from the two molecules in the asymmetric unit are in disallowed regions.

### Example 15

### Data Collection, Structure Determination and Refinement PDE5* with Sildenafil

The structure of the baculovirus engineered PDE5* was solved by molecular replacement (MR) using a combined model of wild-type PDE5 with the structure for the second sub-domain from PDE4 as a search model.

X-ray diffraction data were collected with an RaxisIV image plate detector on an in-house RU200HB rotating anode (Rigaku), with Blue Osmic mirrors (MSC). All data were processed using the HKL package (Otwinowski & Minor, 1997). Data collection statistics are summarised in Table 1b.

The crystals belong to the monoclinic space group P2₁, with unit cell dimensions a=54.93 Å, b=77.77 Å, c=82.05 Å, α=γ=90° β=100.955°. They contain 2 molecules per asymmetric unit (M_{w}=37,562.41 Da) and have a calculated solvent content of 45.87% (V_{M}=2.29; Matthews, 1968).

Molecular replacement was performed using AMORE (CCP4). The resulting map was of good quality and the structure was refitted using QUANTA. Refinement was carried out in the resolution range 30-1.6 Å using CNX (Brunger et al., 1998) with the “mlf” maximum likelihood target function. Partial structure factors from a flat bulk-solvent model and anisotropic B-factor correction were supplied throughout the refinement. The R-factor for the current model is 0.286 (free R-factor, 5% of data, 0.307) for all data in the resolution range 30-1.6 Å. The refinement statistics are summarised in Table 2b.

The current model contains 323 residues per molecule, 537-858 (residue Glu 681A has been numbered to maintain PDE5 numbering scheme). No interpretable electron density is observed for residues: 534, 535 and 536 in molecules A or B. Analysis of the structure using PROCHECK (Laskowski, et al., 1993) shows only two residues from the two molecules in the asymmetric unit are in disallowed regions.

LENGTHY TABLE REFERENCED HERE
US20070015205A1-20070118-T00001
Please refer to the end of the specification for access instructions.

LENGTHY TABLE REFERENCED HERE
US20070015205A1-20070118-T00002
Please refer to the end of the specification for access instructions.

LENGTHY TABLE REFERENCED HERE
US20070015205A1-20070118-T00003
Please refer to the end of the specification for access instructions.

LENGTHY TABLE REFERENCED HERE
US20070015205A1-20070118-T00004
Please refer to the end of the specification for access instructions.

LENGTHY TABLE REFERENCED HERE
US20070015205A1-20070118-T00005
Please refer to the end of the specification for access instructions.

LENGTHY TABLE REFERENCED HERE
US20070015205A1-20070118-T00006
Please refer to the end of the specification for access instructions.

LENGTHY TABLE REFERENCED HERE
US20070015205A1-20070118-T00007
Please refer to the end of the specification for access instructions.

LENGTHY TABLE REFERENCED HERE
US20070015205A1-20070118-T00008
Please refer to the end of the specification for access instructions.

### REFERENCES

Abrahams, J. P. & Leslie, A. (1996) Acta Crystallogr. D 52, 30-42.
Aravind, L. & Ponting, C. P. (1997) TIBS. 22, 458-459.
Brünger, A. et al. (1998), Acta Crystallogr. D 54, 905-921.
Butcher, R. W. & Sutherland, E. W. (1962) J. biol. Chem. 237, 1244-1250.
Charbonneau, H. (1990) in Cyclic Nucleotide Phosphodiesterases: Structure, Regulation and Drug Action (Beavo, J., and Houslay, M. D., eds) pp. 267-296, John Wiley & Sons, Inc., New York.
Charbonneau, H. et al., (1986) Proc. Natl. Acad. Sci. U.S. A., 83, 9308-9312.
Collaborative Computational Project Number 4 (1994) Acta Crystallogr. D 50, 760-763.
Corbin, J. D. & Francis, S. H. (1999) J. Biol. Chem. 274, 13729-13732.
de La Fortelle, E. & Bricogne, G. (1997) Methods Enzymol. 276, 472-494.
Francis, S. H. et al. (1994) J. Biol. Chem. 269, 22477-22480.
Goldberg, N. D. et al (1980) J. Biol. Chem. 255, 10344-10347.
Hendrickson, W. A. et al. (1989) Basic Life Sci. 51 (Synchrotron Radiat. Struct. Biol.), 317-324.
Jiang, H. et al. (1992) J. Biol. Chem. 267, 1015-1019.
Laskowski, R. A. et al. (1993) J. Appl. Crystallogr. 26, 283-291.
Lohman, S. M. et al. (1997) Trends in Biochem. Sci. 22, 307-312.
Martinez, S. et al. (2001) Poster presented at the American Crystallography Association annual meeting, Los Angeles, USA.
Matthews, B. W. (1968) J. Mol. Biol. 33, 491-497.
Nicholls, A. et al. (1993) J. Biophys, A166.
Otwinowski, Z. & Minor, W. (1997) Methods Enzymol. 276, 307-326.
Quanta98, 1998, version 98.1111; Molecular Simulations Inc., San Diego, Calif. 92121-3752.
Rall, T. W. & Sutherland, E. W. (1958) J. Biol. Chem. 232, 1065-1076.
Soderling, S. H. & Beavo, J. A., (2000) Curr. Opin. Cell Biol. 12, 174-179.
Smith, G. D. et al. (1998) Acta Crystallographica Section D-Biological Crystallography. 54 (Pt 5):799-804
Terwilliger, T. C. & Berendzen, J. (1997) Acta Crystallogr. D 53, 571-579 Crystallogr. D 54, 799-804.
Thomas et al. (1990) J. Biol. Chem. 265, 14971-14978.

Xu, R. X. et al. (2000) Science 288, 1822-1825.

| Abbreviations | |
|---|---|
| cAMP | cyclic adenosine monophosphate |
| cGMP | cyclic guanosine monophosphate |
| PDE | phosphodiesterase |
| PGK | protein kinase G |
| MAD | multi-wavelength anomalous dispersion |
| PCR | polymerase chain reaction |
| 2YT | 16 g tryptone, 10 g yeast extract, 5 g NaCl per |
| | litre solution |
| Tris | tris[hydroxymethyl]amino-methane |
| E-64 | epoxysuccinyl-1-leucylamido-(4-guanidino) butane |
| DTT | DL-dithiothreitol |
| β-ME | β-mercaptoethanol |
| IPTG | β-D-isopropyl-thiogalactopyranoside |
| EDTA | ethylenediamine tetraacetic acid |
| Bis-Tris | bis[2-hydroxyethyl]imino-tris[hydroxymethyl]methane |
| PEG | polyethylene glycol |
| PEG2KMME | polyethylene glycol 2000 monomethyl ether |
| PEG4KMME | polyethylene glycol 4000 monomethyl ether |
| PEG8KMME | polyethylene glycol 8000 monomethyl ether |
| rmsd | root mean square deviation |
| rpm | revolutions per minute |
| Moi (or MOI) | multiplicity of infection |
| Sildenafil | 5-[2-ethoxy-5-(4-methyl-1-piperazinylsulphonyl) |
| | phenyl]-1-methyl-3-n-propyl-1,6-dihydro-7H- |
| | pyrazolo [4,3-d]pyrimidin-7-one, which is also known |
| | as 1-[[3-(6,7-dihydro-1-methyl-7-oxo-3-propyl-1H- |
| | pyrazolo[4,3-d]pyrimidin-5-yl)-4-ethoxyphenyl] |
| | sulphonyl]-4-methylpiperazine |
| | (see EP-A-0463756) |
| UK-092,480 | see “Sildenafil” |

LENGTHY TABLE
The patent application contains a lengthy table section. A copy of the table is available in electronic form from the USPTO web site (http://seqdata.uspto.gov/?pageRequest=docDetail&DocID=US20070015205A1) An electronic copy of the table will also be available from the USPTO upon request and payment of the fee set forth in 37 CFR 1.19(b)(3).

1. A crystal of phosphodiesterase 5 (PDE5).

2. The crystal of PDE5 according to claim 1, wherein said PDE5 is from a mammal.

3. The crystal of PDE5 according to claim 1 or claim 2, wherein said PDE5 is from a human.

4. The crystal of PDE5 according to any one of claims 1 to 3, wherein said PDE5 is an isoform selected from the group consisting of PDE5A1, PDE5A2, PDE5A3 and PDE5A4.

5. The crystal of PDE5 according to claim 3 or claim 4, wherein said PDE5 comprises SEQ ID NO: 1 or a homologue, fragment, variant, analogue or derivative thereof.

6. The crystal of PDE5 according to any one of claims 3 to 5, wherein said PDE5 comprises SEQ ID NO: 2 or a homologue, fragment, variant, analogue or derivative thereof.

7. The crystal of PDE5 according to any one of claims 3 to 6, wherein said PDE5 comprises SEQ ID NO: 3 or a homologue, fragment, variant, analogue or derivative thereof.

8. The crystal of PDE5 according to claim 3 or claim 4, wherein said PDE5 comprises SEQ ID NO: 4 or a homologue, fragment, variant, analogue or derivative thereof.

9. The crystal of PDE5 according to any one of claims 3, 4 or 8, wherein said PDE5 comprises SEQ ID NO 5 or a homologue, fragment, variant, analogue or derivative thereof.

10. The crystal of PDE5 according to any one of claims 3, 4, 8 or 9, wherein said PDE5 comprises SEQ ID NO: 6 or a homologue, fragment, variant, analogue or derivative thereof.

11. A crystal of a PDE5/PDE5 ligand complex.

12. The crystal of a PDE5/PDE5 ligand complex according to claim 11, wherein said PDE5 is as defined in any one of claims 5 to 7.

13. The crystal of a PDE5/PDE5 ligand complex according to claim 11, wherein said PDE5 is as defined in any one of claims 8 to 10.

14. The crystal of PDE5 as defined in any one of the claims 5 to 7, which has one or more of the following characteristics:
(a) a space group P6₂;
(b) unit cell dimensions a˜95 Å±1%, b˜95 Å±1%, c˜82 Å±1%, α=β=90°, γ=120°;
(c) 1 molecule per asymmetric unit;
(d) comprises a PDE5 of a molecular weight of approximately 40 kDa±2 kDa;
(e) a calculated solvent content of approximately 43±5%; and
(f) a hexagonal crystal system.

15. The crystal of the PDE5/PDE5 ligand complex according to claim 12, which has one or more of the following characteristics:
(a) a space group P2₁2₁2₁;
(b) unit cell dimensions a˜94 Å±1%, b˜104 Å±1%, c˜142 Å±1%, α=β=γ=90°;
(c) 4 molecules per asymmetric unit;
(d) comprises a PDE5 of a molecular weight of approximately 40 kDa±2 kDa;
(e) a calculated solvent content of approximately 43±5%; and
(f) an orthorhombic crystal system.

16. The crystal of PDE5 as defined in any one of claims 8 to 10 or the crystal of the PDE5/PDE5 ligand complex according to claim 13, which has one or more of the following characteristics:
(a) a space group P2₁;
(b) unit cell dimensions a˜55 Å±1%, b˜78 Å±1%, c˜82 Å±1%, α=γ=90, β˜101°±2°;
(c) 2 molecules per asymmetric unit;
(d) comprises a PDE5 of a molecular weight of approximately 38 kDa±2 kDa;
(e) a calculated solvent content of approximately 46±5%; and
(f) a monoclinic crystal system.

17. The crystal of PDE5 according to any one of claims 5 to 7, wherein said PDE5 has a three-dimensional structure characterised by the atomic co-ordinates set out in Table 3 or a derivative set as expressed in any reference frame.

18. The crystal of the PDE5/PDE5 ligand complex according to claim 12, wherein said PDE5/PDE5 ligand complex has a three-dimensional structure characterised by the atomic co-ordinates set out in Table 4 or a derivative set as expressed in any reference frame.

19. The crystal of PDE5 according to any one of claims 8 to 10, wherein said PDE5 has a three-dimensional structure characterised by the atomic co-ordinates set out in Table 5 or a derivative set as expressed in any reference frame.

20. The crystal of the PDE5/PDE5 ligand complex according to claim 13, wherein said PDE5/PDE5 ligand complex has a three-dimensional structure characterised by the atomic co-ordinates set out in Table 6 or a derivative set as expressed in any reference frame.

21. Use of the atomic co-ordinates determined from the crystal of PDE5 according to claim 17 or the crystal of the PDE5/PDE5 ligand complex according to claim 18 for deriving a three-dimensional structure of (i) a full-length wild-type PDE5 or a mutant, derivative, fragment, variant, analogue or homologue thereof or (ii) a wild-type PDE5 sub-domain or a mutant, derivative, fragment, variant, analogue or homologue thereof.

22. Use of the atomic co-ordinates determined from the crystal of PDE5 according to claim 19 or the crystal of the PDE5/PDE5 ligand complex according to claim 20 for deriving a three-dimensional structure of (i) a full-length wild-type PDE5 or a mutant, derivative, fragment, variant, analogue or homologue thereof or (ii) a wild-type PDE5 sub-domain or a mutant, derivative, fragment, variant, analogue or homologue thereof.

23. Use of the three-dimensional structure of (i) a full-length wild-type PDE5 or a mutant, derivative, fragment, variant, analogue or homologue thereof or (ii) a wild-type PDE5 sub-domain or a mutant, derivative, fragment, variant, analogue or homologue thereof, as derivable according to claim 21 or claim 22 to computationally or otherwise evaluate the binding interactions of a PDE5 ligand with an active site on PDE5.

24. Use according to claim 23, wherein said active site on PDE5 is within the third sub-domain of the protein and is bounded by Helices 15 (H15 813-824) and 14 (H14 772-797), the C-terminus of Helix 13 (H13 749-765), and the C-terminus of Helix 11 (H11 706-721) along with the loop region between Helices 11 and 12a (H12a 725-731) as shown in FIG. 2.

25. Use according to claim 23 or claim 24, wherein said active site on PDE5 comprises Leu 765, Ala 767 and Ile 768 and one or more of Phe 820, Val 782, Phe 786, Tyr 612, Leu 804, Ala 779, Ala 783, Ile 813, Met 816 and Gln 817.

26. Use according to any one of claims 23 to 25 to design a compound capable of associating with PDE5.

27. Use according to any one of claims 23 to 26 to design a compound capable of associating with any active site of PDE5.

28. A method of identifying a compound capable of associating with PDE5, comprising co-crystallising or soaking said compound with the crystal of PDE5 according to any one of claims 1 to 10 and determining the three-dimensional structure to ascertain whether said compound is bound to PDE5.

29. A compound designed by the use according to any one of claims 23 to 27 or identified by the method of claim 28.

30. A method of selecting a PDE5 ligand from a group of potential PDE5 ligands, comprising the following steps:
(a) computationally creating a three-dimensional representation of the structure of PDE5 as derived from the atomic co-ordinates as defined in claim 21 or claim 22, and a three-dimensional representation of the structure of the potential PDE5 ligand;
(b) co-displaying the three-dimensional representation of the potential PDE5 ligand with the three-dimensional representation of the PDE5 structure; and
(c) assessing whether the three-dimensional representation of the potential PDE5 ligand fits the three-dimensional representation of an active site of the PDE5 structure.

31. The method according to claim 30, further comprising the following steps:
(d) incorporating the potential PDE5 ligand in a biological PDE5 activity assay; and
(e) determining whether the potential PDE5 ligand modulates PDE5 activity in said assay.

32. A PDE5 ligand selected by the method of claim 30 or claim 31.

33. Use of a PDE5 ligand according to claim 32 as a pharmaceutical.

34. Use of a PDE5 ligand according to claim 32 in the manufacture of a medicament for the prophylaxis or treatment of a condition, disease, disorder or dysfunction where the inhibition of PDE5 is prophylactically or therapeutically beneficial.

35. Use according to claim 34, wherein said disorder is a mammalian sexual disorder.

36. Use of the atomic co-ordinates determined from the crystal of PDE5 as defined in claim 17 or claim 19 or the crystal of the PDE5/PDE5 ligand complex as defined in claim 18 or claim 20, to solve the crystal structure of a mutant, derivative, fragment, variant, analogue, homologue or complex of a PDE-related protein.

37. Use of the atomic co-ordinates determined from the crystal of PDE5 as defined in claim 17 or claim 19 or the crystal of the PDE5/PDE5 ligand complex as defined in claim 18 or claim 20, to produce a model of the three-dimensional structure of a PDE-related protein.

38. Use of the three-dimensional structure of PDE5 as derivable as set out in claim 21 or claim 22 to design site-directed mutants that mimic other PDE5 isoforms or variants thereof.

39. A crystal of PDE5 or a crystal of a PDE5/PDE5 ligand complex wherein the active site on PDE5 is within the third sub-domain of the protein and is bounded by Helices 15 (H15 813-824) and 14 (H14 772-797), the C-terminus of Helix 13 (H13 749-765), and the C-terminus of Helix 11 (H11 706-721) along with the loop region between Helices 11 and 12a (H12a 725-731) as shown in FIG. 2.

40. A crystal of PDE5 or of a PDE5/PDE5 ligand complex wherein the active site on PDE5 comprises Leu 765, Ala 767 and Ile 768 and one or more of Phe 820, Val 782, Phe 786, Tyr 612, Leu 804, Ala 779, Ala 783, Ile 813, Met 816 and Gln 817.

41. A crystal of PDE5 wherein the crystal system of said crystal is characterised as being monoclinic, orthorhombic or hexagonal.

42. A crystal of a PDE5/PDE5 ligand complex wherein the crystal system of said crystal is characterised as being monoclinic or orthorhombic.

43. A method of producing a structurally stabilised PDE-related protein, comprising:
(a) aligning the amino acid sequence of a PDE-related protein with the amino acid sequence of (i) PDE4, (ii) the catalytic domain of PDE4 as shown in FIG. 1 or (iii) SEQ ID NO: 4;
(b) identifying the structurally equivalent sub-domain of the PDE-related protein that corresponds to SEQ ID NO: 4; and
(c) genetically engineering the replacement of the structurally equivalent sub-domain of the PDE-related protein or a portion thereof with SEQ ID NO: 4 or a homologue, fragment, variant, analogue or derivative thereof.

44. The method according to claim 43, further comprising:
(d) expressing the engineered PDE-related protein from (c) in a host cell.

45. The method according to claim 44, further comprising:
(e) purifying the expressed engineered PDE-related protein from (d).

46. The method according to claim 45, further comprising:
(f) crystallising the purified engineered PDE-related protein from (e).


SEQUENCE LISTING (14 sequences)

SEQ ID NO: 1 - protein, 26 aa, Homo Sapiens
HRGVNNSYIQRSEHPLAQLYCHSIME

SEQ ID NO: 2 - protein, 342 aa, Homo Sapiens
EEETRELQSLAAAVVPSAQTLKITDFSFSDFELSDLETALCTIRMFTDLNLVQNFQMKHEVLCRWILSVKKNYRKNVAYHNWRHAFNTAQCMFAALKAGKIQNKLTDLEILALLIAALSHDLDHRGVNNSYIQRSEHPLAQLYCHSIMEHHHFDQCLMILNSPGNQILSGLSIEEYKTTLKIIKQAILATDLALYIKRRGEFFELIRKNQFNLEDPHQKELFLAMLMTACDLSAITKPWPIQQRIAELVATEFFDQGDRERKELNIEPTDLMNREKKNKIPSMQVGFIDAICLQLYEALTHVSEDCFPLLDGCRKNRQKWQALAEQQEKMLINGESGQAKRN

SEQ ID NO: 3 - protein, 874 aa, Homo Sapiens
ERAGPSFGQQRQQQQPQQQKQQQRDQDSVEAWLDDHWDFTFSYFVRKATREMVNAWFAERVHTIPVCKEGIRGHTESCSCPLQQSPRADNSVPGTPTRKISASEFDRPLRPIVVKDSEGTVSFLSDSEKKEQMPLTPPRFDHDEGDQCSRLLELVKDISSHLDVTALCHKIFLHIHGLISADRYSLFLVCEDSSNDKFLISRLFDVAEGSTLEEVSNNCIRLEWNKGIVGHVAALGEPLNIKDAYEDPRFNAEVDQITGYKTQSILCMPIKNHREEVVGVAQAINKKSGNGGTFTEKDEKDFAAYLAFCGIVLHNAQLYETSLLENKRNQVLLDLASLIFEEQQSLEVILKKIAATIISFMQVQKCTIFIVDEDCSDSFSSVFHMECEELEKSSDTLTREHDANKINYMYAQYVKNTMEPLNIPDVSKDKRFPWTTENTGNVNQQCIRSLLCTPIKNGKKNKVIGVCQLVNKMEENTGKVKPFNRNDEQFLEAFVIFCGLGIQNTQMYEAVERAMAKQMVTLEVLSYHASAAEEETRELQSLAAAVVPSAQTLKITDFSFSDFELSDLETALCTIRMFTDLNLVQNFQMKHEVLCRWILSVKKNYRKNVAYHNWRHAFNTAQCMFAALKAGKIQNKLTDLEILALLIAALSHDLDHRGVNNSYIQRSEHPLAQLYCHSIMEHHHFDQCLMILNSPGNQILSGLSIEEYKTTLKIIKQAILATDLALYIKRRGEFFELIRKNQFNLEDPHQKELFLAMLMTACDLSAITKPWPIQQRIAELVATEFFDQGDRERKELNIEPTDLMNREKKNKIPSMQVGFIDAICLQLYEALTHVSEDCFPLLDGCRKNRQKWQALAEQQEKMLINGESGQAKRN

SEQ ID NO: 4 - protein, 27 aa, Homo Sapiens
HPGVSNQFLINTNSELALMYNDESVLE

SEQ ID NO: 5 - protein, 326 aa, Homo Sapiens
EEETRELQSLAAAVVPSAQTLKITDFSFSDFELSDLETALCTIRMFTDLNLVQNFQMKHEVLCRWILSVKKNYRKNVAYHNWRHAFNTAQCMFAALKAGKIQNKLTDLEILALLIAALSHDLDHPGVSNQFLINTNSELALMYNDESVLEHHHFDQCLMILNSPGNQILSGLSIEEYKTTLKIIKQAILATDLALYIKRRGEFFELIRKNQFNLEDPHQKELFLAMLMTACDLSAITKPWPIQQRIAELVATEFFDQGDRERKELNIEPTDLMNREKKNKIPSMQVGFIDAICLQLYEALTHVSEDCFPLLDGCRKNRQKWQALAE

SEQ ID NO: 6 - protein, 858 aa, Homo Sapiens
ERAGPSFGQQRQQQQPQQQKQQQRDQDSVEAWLDDHWDFTFSYFVRKATREMVNAWFAERVHTIPVCKEGIRGHTESCSCPLQQSPRADNSVPGTPTRKISASEFDRPLRPIVVKDSEGTVSFLSDSEKKEQMPLTPPRFDHDEGDQCSRLLELVKDISSHLDVTALCHKIFLHIHGLISADRYSLFLVCEDSSNDKFLISRLFDVAEGSTLEEVSNNCIRLEWNKGIVGHVAALGEPLNIKDAYEDPRFNAEVDQITGYKTQSILCMPIKNHREEVVGVAQAINKKSGNGGTFTEKDEKDFAAYLAFCGIVLHNAQLYETSLLENKRNQVLLDLASLIFEEQQSLEVILKKIAATIISFMQVQKCTIFIVDEDCSDSFSSVFHMECEELEKSSDTLTREHDANKINYMYAQYVKNTMEPLNIPDVSKDKRFPWTTENTGNVNQQCIRSLLCTPIKNGKKNKVIGVCQLVNKMEENTGKVKPFNRNDEQFLEAFVIFCGLGIQNTQMYEAVERAMAKQMVTLEVLSYHASAAEEETRELQSLAAAVVPSAQTLKITDFSFSDFELSDLETALCTIRMFTDLNLVQNFQMKHEVLCRWILSVKKNYRKNVAYHNWRHAFNTAQCMFAALKAGKIQNKLTDLEILALLIAALSHDLDHPGVSNQFLINTNSELALMYNDESVLEHHHFDQCLMILNSPGNQILSGLSIEEYKTTLKIIKQAILATDLALYIKRRGEFFELIRKNQFNLEDPHQKELFLAMLMTACDLSAITKPWPIQQRIAELVATEFFDQGDRERKELNIEPTDLMNREKKNKIPSMQVGFIDAICLQLYEALTHVSEDCFPLLDGCRKNRQKWQALAE

SEQ ID NO: 7 - DNA, 34 bp, Homo Sapiens
cgtgaattcatggaggaagaaacaagagagctac

SEQ ID NO: 8 - DNA, 42 bp, Homo Sapiens
cgttctagactatcagttccgcttggcctggccgctttcccc

SEQ ID NO: 9 - DNA, 79 bp, Homo Sapiens
cgtgaattcatgcatcatcatcatcatcatcttctggttccgcgtggatctgcgcccgaggaagaaacaagagagctac

SEQ ID NO: 10 - DNA, 72 bp, Homo Sapiens
caaagaaagttctgaatttgtgttgatgagaaactgattggagactccaggatgatccaaatcgtggcttag

SEQ ID NO: 11 - DNA, 72 bp, HomoSapiens
atcaacacaaattcagaacttgctttgatgtataatgatgaatctgtgttggaacaccatcattttgaccag

SEQ ID NO: 12 - DNA, 39 bp, Homo Sapiens
cgttctagactatcattctgcaagggcctgccatttctg

SEQ ID NO: 13 - DNA, 31 bp, Homo Sapiens
cgtcatatggaggaagaaacaagagagctac

SEQ ID NO: 14 - DNA, 39 bp, Homo Sapiens
cgtctcgagctatcattctgcaagggcctgccatttctg